(19) United States
(12) Patent Application Publication
(10) Pub. No.: US 2025/0232468 A1
(43) Pub. Date: Jul. 17, 2025

(54) DEVICE AND METHOD FOR GUIDING TRANS-CATHETER AORTIC VALVE REPLACEMENT PROCEDURE

(71) Applicant: Libra Science Ltd., Road Town, Tortola (VG)

(72) Inventors: Shlomo BEN-HAIM, Milan (IT); Barouch Asi ELAD, Kiryat Ono (IL)

(73) Assignee: Libra Science Ltd., Road Town, Tortola (VG)

(21) Appl. No.: 18/853,728

(22) PCT Filed: Apr. 3, 2023
(86) PCT No.: PCT/IB2023/053359

§ 371 Date: Oct. 3, 2024

Related U.S. Application Data
(60) Provisional application No. 63/356,076, filed on Jun. 28, 2022.
(60) Provisional application No. 63/327,377, filed on Apr. 5, 2022.

(51) Int. Cl. G06T 7/73; A61B 34/10; G06T 5/60; G06T 7/00; G06T 7/60; G16H 30/40

(52) U.S. Cl. CPC G06T 7/73; A61B 34/10; G06T 5/60; G06T 7/0012; G06T 7/60; G16H 30/40; A61B 2034/107; G06T 2207/10121; G06T 2207/20081; G06T 2207/30052; G06T 2207/30204

(57) ABSTRACT

Disclosed is a method for assisting a surgeon in bringing a medical implement having a marker to a target location within a body of a patient. According to some embodiments, the method includes receiving a first image of the medical implement on its way to the target location in the body of the patient; processing the image to obtain an enhanced image showing at least said marker; and providing the original image and the enhanced image for display to the surgeon, wherein the enhanced image is provided for display as an inset on a display of the first image.

### RELATED APPLICATIONS

This application claims the benefit of priority of U.S. Provisional Patent Application No. 63/356,076 filed on Jun. 28, 2022, and U.S. Provisional Patent Application No. 63/327,377 filed on Apr. 5, 2022 the contents of which are incorporated herein by reference in their entirety.

### FIELD OF THE INVENTION

The present invention, in some embodiments therefore, relates to the field of image processing for assisting physicians in carrying out medical interventions, and more particularly, but not exclusively, for image processing for reducing cognitive load from a physician carrying out a trans-catheter intervention.

### BACKGROUND OF THE INVENTION

Currently, implantable medical devices, such as large stents, scaffolds, and other cardiac intervention devices are utilized to repair or replace problem native biological systems. For example, heart valve replacement in patients with severe valve disease is a common surgical procedure. The replacement can conventionally be performed by open heart surgery, in which the heart is usually arrested and the patient is placed on a heart bypass machine. In recent years, prosthetic heart valves have been developed which are implanted using minimally invasive procedures such as transapical or percutaneous approaches. These procedures involve compressing the prosthetic heart valve radially to reduce its diameter, inserting the prosthetic heart valve into a delivery device, such as a catheter, and advancing the delivery device to the correct anatomical position in the heart. Once properly positioned, the prosthetic heart valve is deployed by radial expansion within the native valve annulus. Since these procedures are based on minimally invasive approaches where catheters are inserted into the body, physicians cannot directly visualize the procedure taking place as in open heart surgery. As such, physicians rely on imaging modalities designed to capture pictures within the body, such as x-rays, for guiding these procedures. Radiography markers, which are easier to see on the x-rays, may be placed on the medical devices inserted into the body.

Examples of patent applications describing radiographic markers for assisting a physician in proper placement of a prosthetic heart valve include US20210275299, US20140330372, and US 20220061985 to Medtronic, US20100249908 and WO22046585 to Edwards Lifesciences Corporation, and US20200352716 to Icahn School of Medicine At Mount Sinai.

### SUMMARY OF THE INVENTION

An aspect of some embodiments of the present disclosure includes a computer implemented method for generating a presentation of at least one image for assisting an operator (e.g., surgeon) in bringing a medical implement having a marker to a target location within a body of a patient, the method comprising:
receiving a first image of the medical implement in the body of the patient, the implement being on its way to the target location;
processing the first image to obtain an enhanced image showing at least said marker; and
providing the first image and the enhanced image for display, wherein the enhanced image is provided for display as an inset on a display of the first image.

An aspect of some embodiments of the present invention includes a computer implemented method for generating a presentation of at least one image for assisting an operator (e.g., surgeon) in bringing a medical implement having a marker to a target location within a body of a patient, the method comprising:
receiving a first image of the medical implement in the body of the patient, the implement being on its way to the target location;
processing the first image to obtain an enhanced image of a region of interest showing at least said marker; and
providing the enhanced image for display,
   wherein the processing comprises inputting the first image to a machine-learning model trained to identify the region of interest encompassing at least said marker.

Embodiments of both aspects may be characterized in that the processing comprises
identifying a portion of the first image as a region of interest comprising the marker;
cropping the identified portion from the first image; and
enhancing the cropped portion of the first image.

In some embodiments, the enhanced image shows a portion of the medical implement enlarged in comparison to its size in the first image.

In some embodiments, the method further includes estimating a roll angle of the medical implement based on appearance of the marker in the first or enhanced image, and
providing for display an indication to the estimated roll angle.

Optionally, the marker is shaped to display on the first image a portion of its length depending on the roll angle, and the processing comprises estimating the roll angle based on the length of the marker shown in the first or enhanced image.

Alternatively or additionally, estimating the roll angle comprises identifying spatial relationships between marking elements of the marker shown in the first or enhanced image.

In some embodiments, the processing comprises inputting the first image to a machine-learning model trained to identify the region of interest encompassing at least said marker.

In some embodiments, the processing comprises inputting the first image to a machine-learning model trained to estimate the roll angle.

In some embodiments, the method is repeated at least 10 times per second, to provide for display of a cine of enhanced images.

In some embodiments, the first image is a fluoroscopic image.

An aspect of some embodiments of the present disclosure includes a system for generating a presentation of at least one image for assisting an operator (e.g., surgeon) in bringing a medical implement having a marker to a target location within a body of a patient, the system comprising:
a memory, storing instructions;
a processor, configured to execute the instructions,
   wherein executing the instructions cause the processor to
receive a first image of the medical implement;
process the first image to obtain an enhanced image showing at least said marker; and
provide the first image and the enhanced image for display, wherein the enhanced image is provided for display as an inset on a display of the first image.

An aspect of some embodiments of the present disclosure includes a system for generating a presentation of at least one image for assisting an operator (e.g., surgeon) in bringing a medical implement having a marker to a target location within a body of a patient, the system comprising
a memory, storing instructions; and
a processor, configured to execute the instructions,
   wherein executing the instructions cause the processor to
receive a first image of the medical implement in the body of the patient, the implement being on its way to the target location;
apply to the first image a machine-learning model trained to identify a region of interest encompassing at least said marker;
process the first image to obtain an enhanced image of the region of interest showing at least said marker; and
provide the enhanced image for display to the operator.

Embodiments of the latter two aspects may be characterized in that the instructions cause the processor to
identify a portion of the first image as a region of interest comprising the marker;
crop the identified portion from the first image; and
enhance at least the cropped portion of the first image.

In some embodiments, the enhanced image shows a portion of the medical implement enlarged in comparison to a size of said portion of the medical implement in the first image.

In some embodiments, the instructions further cause the processor to
estimate a roll angle of the medical implement based on the appearance of the mark in the first or enhanced image, and
provide for display an indication to the estimated roll angle.

Optionally, the instructions cause the processor to estimate the roll angle based on the length of the marker shown in the first or enhanced image.

Alternatively or additionally, the instructions cause the processor to estimate the roll angle by identifying spatial relationships between marking elements of the marker shown in the first or enhanced image.

In some embodiments, the instructions cause the processor to apply, to the first image or to the enhanced image, a machine-learning model trained to identify the region of interest encompassing at least said marker.

In some embodiments, the instructions cause the processor to apply, to the first image or to the enhanced image, a machine-learning model trained to estimate the roll angle.

In some embodiments, the instructions cause the processor to repeat the receiving, processing, and providing for display at least 10 times per second, to provide for display of a cine of enhanced images.

In some embodiments, the first image is a fluoroscopic image.

Some embodiments further include an input for receiving the first image from an imaging device and said instructions cause the processor to receive the image via said input.

Optionally, the instructions cause the processor to provide the enhanced image for display to an output connected to a display of the imaging device.

An aspect of some embodiments of the present disclosure includes a computer implemented method for computing a state of a marker of a device for guiding a trans-catheter aortic valve replacement intervention in a patient, comprising:
obtaining images (e.g., fluoroscopic images) capturing an aortic valve prosthesis device in the aorta of the patient;
feeding at least one of the obtained images to a machine-learning model trained to identify the state of a marker on the valve prosthesis device;
receiving from the machine-learning model output indicating the state of the marker; and
displaying the indication of the state of the marker according to the received output of the machine-learning model.

In some embodiments, the method comprises repeating in a plurality of iterations, the obtaining, feeding, and receiving, and further comprising displaying a state-change indication, indicative to a change in the output.

In some embodiments, the marker is configured to be aligned with a native commissure of a native heart valve of the patient, and the state of the marker indicates whether the marker is aligned with the native commissure.

In some embodiments, the marker is composed of a plurality of marking units, the spatial relations between which are indicative to the orientation being proper or not, and the indication of the state indicates whether the orientation is proper or not.

In some embodiments, the method further comprises receiving input indicative to the fluoroscopic view at which the image has been taken, and feeding the fluoroscopic view into the machine-learning model in combination with the at least one of the obtained images.

In some embodiments, the output of the machine-learning model indicates if the orientation of the device is proper or not, based on the input of the fluoroscopic view and the at least one of the obtained images.

In some embodiments, the indication of the state of the marker comprises an indication of an orientation of the aortic valve prosthesis device.

In some embodiments, the marker comprises three markers spaced apart along a circumference of the aortic valve prosthesis device, the orientation of the aortic valve prosthesis is selected from a group consisting of: two markers overlap on a left side and a third marker does not overlap, two markers overlap on a right side and a third marker does not overlap, and none of the three markers are overlapping.

In some embodiments, each one of the three markers is placed at a commissures of the aortic valve prosthesis device.

In some embodiments, the marker includes a single main marker, and the orientation indicates the location of the single main marker, selected from a group consisting of: outer, inner, and central. In some embodiments, the orientation is selected from a group including: correct orientation and incorrect orientation.

In some embodiments, correct orientation denotes commissures of the prosthetic valve are non-aligned with the coronary ostia, and incorrect orientation denotes commissures of the prosthetic valve are aligned with the coronary ostia.

In some embodiments, a same orientation of the prosthetic aortic valve is detected from different poses of the marker.

In some embodiments, the method further comprises determining a target pose of an imaging sensor capturing a target fluoroscopic image for which the indication of the state of the marker is obtained, obtaining a second fluoroscopic image captured by the imaging sensor at a second pose different than the target pose, wherein the indication of the state of the marker at the second pose is non-determinable or determinable with a lower accuracy than for the target pose, computing a transformation function for transforming an image from the second pose to the target pose, and applying the transformation function to at least a portion of the second fluoroscopic image depicting the marker for obtaining a transformed image depicting the marker at the target pose.

An aspect of some embodiments of the present disclosure include an apparatus of computing a state of a marker of a device for guiding a trans-catheter aortic valve replacement intervention in a patient, comprising
a processor; and
a digital memory storing instructions,
wherein when executed by the processor, the instructions cause the processor to
   obtain 2D images (e.g., fluoroscopic 2D images) from an imaging device (e.g., fluoroscopic imaging device) capturing an aortic valve prosthesis device in the aorta of the patient during the intervention;
   feed at least one of the obtained images to a machine-learning model trained to identify the state of a marker on the valve prosthesis device;
   receive from the machine-learning model output indicating the state of the marker; and
   cause display of the indication of the state of the marker according to the received output.

In some embodiments, the instructions cause the processor to repeatedly in a plurality of iterations, obtain images, feed them to the machine-learning model, receive a status indication for each respective image, and cause display of a status change indication when the status indication changes within the plurality of iterations.

In some embodiments, the marker is composed of a plurality of marking units, the spatial relations between which are indicative to the state of the marker.

In some embodiments, the apparatus further comprises a display device, and wherein the instructions cause the processor to cause the display of the status indication using the display device.

In some embodiments, the instructions cause the processor to display a visual indication to the status indication received as output from the machine-learning model.

In some embodiments, the instructions cause the processor to display an audio indication to the status indication received as output from the machine-learning model.

In some embodiments, the instructions cause the processor to repeat in a plurality of iterations, the obtaining, feeding, and receiving, and further comprising displaying a state-change indication, indicative to a change in the output of the machine-learning model within the plurality of iterations.

In some embodiments, the marker is configured to be aligned with a native commissure of a native heart valve of the patient, and the state of the marker indicates whether the marker is aligned with the native commissure.

In some embodiments, the marker is composed of a plurality of marking units, the spatial relations between which are indicative to the orientation being proper or not, and the indication of the state indicates whether the orientation is proper or not.

In some embodiments, the instructions cause the processor to receive input indicative to the fluoroscopic view at which the image has been taken, and feeding the fluoroscopic view into the machine-learning model in combination with the at least one of the obtained images.

In some embodiments, the output of the machine-learning model indicates if the orientation of the device is proper or not, based on the input of the fluoroscopic view and the at least one of the obtained images.

In some embodiments, the indication of the state of the marker comprises an indication of an orientation of the aortic valve prosthesis device.

In some embodiments, the marker comprises three markers spaced apart along a circumference of the aortic valve prosthesis device, the orientation of the aortic valve prosthesis is selected from a group consisting of: two markers overlap on a left side and a third marker does not overlap, two markers overlap on a right side and a third marker does not overlap, and none of the three markers are overlapping.

In some embodiments, each one of the three markers is placed at a commissures of the aortic valve prosthesis device.

In some embodiments, the marker includes a single main marker, and the orientation indicates the location of the single main marker, selected from a group consisting of: outer, inner, and central. In some embodiments, the orientation is selected from a group including: correct orientation and incorrect orientation.

In some embodiments, correct orientation denotes commissures of the prosthetic valve are non-aligned with the coronary ostia, and incorrect orientation denotes commissures of the prosthetic valve are aligned with the coronary ostia.

In some embodiments, a same orientation of the prosthetic aortic valve is detected from different poses of the marker.

In some embodiments, the instructions cause the processor to determine a target pose of an imaging sensor capturing a target fluoroscopic image for which the indication of the state of the marker is obtained; obtain a second fluoroscopic image captured by the imaging sensor at a second pose different than the target pose, wherein the indication of the state of the marker at the second pose is non-determinable or determinable with a lower accuracy than for the target pose; compute a transformation function for transforming an image from the second pose to the target pose; and apply the transformation function to at least a portion of the second fluoroscopic image depicting the marker for obtaining a transformed image depicting the marker at the target pose.

An aspect of some embodiments of the present disclosure include a computer-implemented method of training a ML model for determining an orientation of an aortic valve prosthesis for trans-catheter deployment depicted in a medical image, comprising:
for each sample medical image of a plurality of sample original medical images (also referred herein as sample original images) of a plurality of subjects, wherein a sample original medical image depicts the aortic valve prosthesis with a marker:
   defining a region of interest (ROI) of the sample original medical image that depicts the marker;
   creating an enhanced medical image from the ROI;
   determining an orientation of the aortic valve prosthesis depicted in the enhanced medical image;
   creating a record comprising the sample original medical image, and a ground truth indicating the orientation;
creating a training dataset comprising a plurality of records; and
training the ML model on the training dataset for generating an outcome of the orientation in response to an input of a target original image depicting a target aortic valve prosthesis for trans-catheter deployment.

In some embodiments, the ground truth of the record further includes the enhanced medical image, and the outcome of the ML model further includes the enhanced medical image.

In some embodiments, the enhanced medical image is of a higher quality than the ROI of the sample medical image.

In some embodiments, the enhanced medical image is an enlargement of the ROI of the sample medical image.

In some embodiments, the ROI is a frame having dimensions smaller than the sample medical image, the ROI sized for depicting the marker, at least a portion of the aortic valve prosthesis, and tissues in proximity to the aortic valve prosthesis.

In some embodiments, the orientation of the medical image is selected from a group comprising: whether the orientation of the medical device is proper or not, whether the marker is aligned with the native commissure of the aortic annulus where the aortic valve prosthesis is to be deployed, a roll angle, and a classification category.

Optionally, the classification category is selected from a group consisting of: inner curve state, outer curve state, and middle state.

In some embodiments, the method further comprises obtaining a pose of an imager that captured the sample medical image, wherein the record includes the pose and wherein the ML model generates the outcome in response to a further input of the pose.

Optionally, the pose is obtained by applying an optical character recognition process to the sample medical image, and extracting the automatically recognized characters.

In some embodiments, creating the training dataset comprises creating a first training dataset comprising a plurality of first records, each first record including the sample original medical image and a ground truth of the enhanced medical image, creating a second training dataset comprising a plurality of second records, each second record including the enhanced medical image and a ground truth of the orientation, wherein the ML model comprises a first ML model component and a second ML model component, and training comprises: training the first ML model component on the first training dataset for generating an outcome of the enhanced medical image in response to the input of the target original image, training second ML model component on the second training dataset for generating an outcome of the orientation in response to an input of the enhanced medical image generated by the first ML model.

In some embodiments, the ROI of the sample original medical image comprises a first boundary encompassing an entirety of the aortic valve prosthesis, and wherein the enhanced medical image comprises a portion of the sample original image within a second boundary located within the first boundary, the second boundary encompassing the marker and a portion of the aortic valve prosthesis in proximity to the marker and excluding a remainder of the aortic valve prosthesis.

In some embodiments, creating the training dataset comprises: creating a first training dataset comprising a plurality of first records, each first record including the sample original medical image and a ground truth of the first boundary encompassing an entirety of the aortic valve prosthesis, creating a second training dataset comprising a plurality of second records, each second record including the portion of the sample original image within first boundary and a ground truth of the second boundary, creating a third training dataset comprising a plurality of third records, each third record including a portion of the sample original image within the second boundary and a ground truth of the orientation, wherein the ML model comprises a first ML model component, a second ML model component, and a third ML component, and training comprises: training the first ML model component on the first training dataset for generating an outcome of the first boundary in response to the input of the target original image, training the second ML model component on the second training dataset for generating an outcome of the second boundary in response to the input of the first boundary generated by the first ML, and training the third ML model component on the third training dataset for generating an outcome of the orientation in response to an input of the portion of the target original medical image within the second boundary generated by the second ML model.

An aspect of some embodiments of the present disclosure include a system for training a ML model for determining an orientation of an aortic valve prosthesis for trans-catheter deployment depicted in a medical image, the system comprising: a memory, storing instructions; and a processor, configured to execute the instructions, wherein executing the instructions cause the processor to: for each sample medical image of a plurality of sample original medical images of a plurality of subjects, wherein a sample original medical image depicts the aortic valve prosthesis with a marker: define a region of interest (ROI) of the sample original medical image that depicts the marker; create an enhanced medical image from the ROI; determine an orientation of the aortic valve prosthesis depicted in the enhanced medical image; create a record comprising the sample original medical image, and a ground truth indicating the orientation; create a training dataset comprising a plurality of records; and train the ML model on the training dataset for generating an outcome of the orientation in response to an input of a target original image depicting a target aortic valve prosthesis for trans-catheter deployment.

In some embodiments, the ground truth of the record further includes the enhanced medical image, and the outcome of the ML model further includes the enhanced medical image.

In some embodiments, the enhanced medical image is of a higher quality than the ROI of the sample medical image.

In some embodiments, the enhanced medical image is an enlargement of the ROI of the sample medical image.

In some embodiments, the ROI is a frame having dimensions smaller than the sample medical image, the ROI sized for depicting the marker, at least a portion of the aortic valve prosthesis, and tissues in proximity to the aortic valve prosthesis.

In some embodiments, the orientation of the medical image is selected from a group comprising: whether the orientation of the medical device is proper or not, whether the marker is aligned with the native commissure of the aortic annulus where the aortic valve prosthesis is to be deployed, a roll angle, and a classification category.

In some embodiments, the classification category is selected from a group consisting of: inner curve state, outer curve state, and middle state.

In some embodiments, the instructions further cause the processor to obtain a pose of an imager that captured the sample medical image, wherein the record includes the pose and wherein the ML model generates the outcome in response to a further input of the pose.

In some embodiments, the pose is obtained by applying an optical character recognition process to the sample medical image, and extracting the automatically recognized characters.

In some embodiments, creating the training dataset comprises creating a first training dataset comprising a plurality of first records, each first record including the sample original medical image and a ground truth of the enhanced medical image, creating a second training dataset comprising a plurality of second records, each second record including the enhanced medical image and a ground truth of the orientation, wherein the ML model comprises a first ML model component and a second ML model component, and training comprises: training the first ML model component on the first training dataset for generating an outcome of the enhanced medical image in response to the input of the target original image, training second ML model component on the second training dataset for generating an outcome of the orientation in response to an input of the enhanced medical image generated by the first ML model.

In some embodiments, the ROI of the sample original image comprises a first boundary encompassing an entirety of the aortic valve prosthesis, and wherein the enhanced medical image comprises a portion of the sample original image within a second boundary located within the first boundary, the second boundary encompassing the marker and a portion of the aortic valve prosthesis in proximity to the marker and excluding a remainder of the aortic valve prosthesis.

In some embodiments, creating the training dataset comprises: creating a first training dataset comprising a plurality of first records, each first record including the sample original medical image and a ground truth of the first boundary encompassing an entirety of the aortic valve prosthesis, creating a second training dataset comprising a plurality of second records, each second record including the portion of the sample original image within first boundary and a ground truth of the second boundary, creating a third training dataset comprising a plurality of third records, each third record including a portion of the sample original image within the second boundary and a ground truth of the orientation, wherein the ML model comprises a first ML model component, a second ML model component, and a third ML component, and training comprises: training the first ML model component on the first training dataset for generating an outcome of the first boundary in response to the input of the target original image, training the second ML model component on the second training dataset for generating an outcome of the second boundary in response to the input of the first boundary generated by the first ML, and training the third ML model component on the third training dataset for generating an outcome of the orientation in response to an input of the portion of the target original medical image within the second boundary generated by the second ML model.

An aspect of some embodiments of the present disclosure include a computer-implemented method of generating a presentation for guiding a trans-catheter aortic valve implantation (TAVI) medical procedure, comprising: feeding an original medical image depicting at least a portion of an aortic valve prosthesis with marker for trans-catheter deployment into a machine learning (ML) model, obtaining an enhanced image comprising a ROI of the original medical image that depicts the marker and the at least portion of the aortic valve prosthesis, and an orientation of the aortic valve prosthesis, and generating instructions for presenting the enhanced image as an inset of the original medical image presented on display, and for presenting the orientation of the aortic valve prosthesis.

An aspect of some embodiments of the present disclosure include a system for generating a presentation for guiding a trans-catheter aortic valve implantation (TAVI) medical procedure, comprising: a memory, storing instructions, a processor, configured to execute the instructions, wherein executing the instructions cause the processor to feed an original medical image depicting at least a portion of an aortic valve prosthesis with marker for trans-catheter deployment into a ML model, obtain an enhanced image comprising a ROI of the original medical image that depicts the marker and the at least the portion of the aortic valve prosthesis, and an orientation of the aortic valve prosthesis, and generate instructions for presenting the enhanced image as an inset of the target original medical image presented on display, and for presenting the orientation of the aortic valve prosthesis.

An aspect of some embodiments of the present disclosure include a computer implemented method for estimating a roll angle of a medical implement having a marker, when delivered to a target location within a body of a patient, the method comprising: receiving an image of the medical implement in the body of the patient, the implement being on its way to the target location, estimating a roll angle of the medical implement based on appearance of the marker in the image; wherein the estimating comprises inputting the first image to a machine-learning model trained to identify the roll angel, and providing for display an indication to the estimated roll angle.

An aspect of some embodiments of the present disclosure include a system for estimating a roll angle of a medical implement having a marker, when delivered to a target location within a body of a patient, the system comprising: a memory, storing instructions, a processor, configured to execute the instructions, wherein executing the instructions cause the processor to receive an image of the medical implement in the body of the patient, the implement being on its way to the target location, estimate a roll angle of the medical implement based on appearance of the marker in the image; wherein the estimating comprises inputting the first image to a machine-learning model trained to identify the roll angel, and provide for display an indication to the estimated roll angle.

An aspect of some embodiments of the present disclosure include a computer implemented method of computing a state of a marker of a medical implement for guiding a medical procedure in a patient, comprising: obtaining images capturing the medical implement in the body of the patient, feeding at least one of the obtained images to a machine-learning model trained to identify a state of a marker on the medical implement, receiving from the machine-learning model, output indicating the state of the marker, and displaying the indication of the state of the marker according to the received output.

An aspect of some embodiments of the present disclosure include a system for computing a state of a marker of a medical implement for guiding a medical procedure in a patient, the system comprising: a memory, storing instructions, a processor, configured to execute the instructions, wherein executing the instructions cause the processor to: obtain images capturing the medical implement in the body of the patient, feed at least one of the obtained images to a machine-learning model trained to identify a state of a marker on the medical implement, receive from the machine-learning model, output indicating the state of the marker, and display the indication of the state of the marker according to the received output.

An aspect of some embodiments of the present disclosure include a computer implemented method of computing a state of a marker of a device for guiding a trans-catheter aortic valve replacement intervention in a patient, comprising: obtaining images capturing an aortic valve prosthesis device in the aorta of the patient, feeding at least one of the obtained images to a machine-learning model trained to identify a state of a marker on the valve prosthesis device, receiving from the machine-learning model, output indicating the state of the marker, and displaying the indication of the state of the marker according to the received output.

An aspect of some embodiments of the present disclosure include a system for computing a state of a marker of a device for guiding a trans-catheter aortic valve replacement intervention in a patient, comprising: a memory, storing instructions, a processor, configured to execute the instructions, wherein executing the instructions cause the processor to: obtain images capturing an aortic valve prosthesis device in the aorta of the patient, feed at least one of the obtained images to a machine-learning model trained to identify a state of a marker on the valve prosthesis device, receive from the machine-learning model, output indicating the state of the marker, and display the indication of the state of the marker according to the received output.

An aspect of some embodiments of the present disclosure include a computer-implemented method of generating a presentation for guiding a medical procedure, comprising: feeding an original medical image depicting at least a portion of medical implement with one or more markers for trans-catheter deployment into a ML model, obtaining an enhanced image comprising a ROI of the original medical image that depicts the one or more markers and the at least the portion of the medical implement, and an orientation of the medical implement, and generating instructions for presenting the enhanced image as an inset of the original medical image presented on display, and for presenting the orientation of the medical implement.

An aspect of some embodiments of the present disclosure include a system for generating a presentation for guiding a medical procedure, comprising: a memory, storing instructions, a processor, configured to execute the instructions, wherein executing the instructions cause the processor to: feed an original medical image depicting at least a portion of medical implement with one or more markers for trans-catheter deployment into a ML model, obtain an enhanced image comprising a ROI of the original medical image that depicts the one or more markers and the at least the portion of the medical implement, and an orientation of the medical implement, and generate instructions for presenting the enhanced image as an inset of the original medical image presented on display, and for presenting the orientation of the medical implement.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 is a pictorial illustration of an operating room equipped with a system for guiding medical procedure, in accordance with some embodiments of the present invention;

FIG. 2 is an exemplary display of an original fluoroscopic image displayed with an enhanced image as an inset, in accordance with some embodiments of the present invention;

FIG. 3 is a simplified flowchart of a method for generating one or more images for assisting an operator in bringing a medical implement to a target location within a body of a patient, in accordance with some embodiments of the present invention;

FIG. 4 is a simplified block diagram of a system configured to carry out the method of FIG. 3 and/or FIG. 5 in accordance with some embodiments of the present invention;

FIG. 5 is a simplified flowchart of a method of training ML model(s) for generating an outcome of an orientation of a medical device and/or an enhanced image, in accordance with some embodiments of the present invention;

FIG. 6 is a schematic depicting exemplary orientations of an aortic valve prosthesis with three spaced apart markings, in accordance with some embodiments of the present invention;

FIG. 7 includes schematics of different orientations of an aortic valve prosthesis that includes three markers located at the commissures of the aortic valve prosthesis, in accordance with some embodiments of the present invention;

FIG. 8 includes schematics of different orientations of an aortic valve prosthesis that includes a single main marker, in accordance with some embodiments of the present invention;

FIG. 9 is a schematic depicting a sample original image, a first boundary box, a second boundary box, and an enhanced image which is labelled with the ground truth of orientation of the valve prosthesis according to the depicted marker, in accordance with some embodiments of the present invention; and

FIG. 10 is a schematic of an exemplary neural network architecture of ML model(s), in accordance with some embodiments of the present invention.

### DETAILED DESCRIPTION OF EMBODIMENTS OF THE INVENTION

The present invention, in some embodiments therefore, relates to the field of image processing for assisting an operator (e.g., surgeon) in bringing a medical implement to a target location within a body of a patient. More particularly, but not exclusively, embodiments of the invention generate images and/or compute indications based on analysis of images for assisting the operator in bringing the medical implement to the target location oriented in a predetermined, desired, orientation.

As used herein, the term medical implement may sometimes be interchanged with the term medical device. The term medical implement and/or medical device may sometimes refer to the aortic valve prosthesis device described herein, but is not necessarily limited to the aortic valve prosthesis device described herein. The term medical implement and/or medical device may sometimes be interchanged with the term aortic valve prosthesis device. The term aortic valve prosthesis may sometimes be used as a not necessarily limiting example of the medical implement and/or medical device. The medical device referred to herein may include medical devices for implantation (referred to as medical implement) or other devices used in minimally invasive medical procedures (e.g., intra-body catheter or balloon). The medical device referred to herein may include other devices designed for trans-catheter interventions, optionally delivered in a compressed state for expansion at the target side, for example, devices for closure (of atrial septal defects (ASD), patent foramen ovale (PFO), devices for ablation, and the like.

As used herein, the term marker refers to a physical component made of a material designed to be visually apparent on images, for example, a radio opaque marker designed to be visually apparent on x-ray images. The term marker may refer to multiple markers.

As used herein, embodiments that are based on machine-learning (ML) models are not necessarily limiting, for example, unless explicitly claimed and/or described. For example, approaches for generating training datasets for training the ML model are related to the ML model. The ML model may be an example, and sometimes other image processing approaches may be used, as described herein. For example, different approaches to identify the marker and/or ROI encompassing the marker may be described.

As used herein, the terms image, medical image, 2D medical image, 2D image, fluoroscopic image, and 2D fluoroscopic image, may sometimes be interchanged. Fluoroscopic images may sometimes serve as an example of images, in cases where other types of images may be used.

As used herein, the term image and medical image are used interchangeably. For example, original medical image and original image are used interchangeably. In another example, the terms sample original medical image and sample original image are used interchangeably.

As used herein, the terms imaging device, medical imaging device, imaging sensor, and imager, are used interchangeably.

As used herein, the terms heart valve, valve prosthesis device, prosthetic valve, prosthetic aortic valve, aortic prosthetic valve, aortic valve prosthesis, aortic valve prosthesis device, prosthetic heart valve, aortic valve implant, valve, and prosthesis, are used interchangeably. The aforementioned terms may sometimes be interchanged with the term medical implement and/or medical device. The aforementioned terms may sometimes serve as an example of a medical implement and/or medical device, and other intra-body medical devices including markers in which the orientation of the intra-body medical is required may be referred to.

As used herein, the term medical implement and/or medical device may refer to any intra-body device with markers in which the orientation of the intra-body device is to be known.

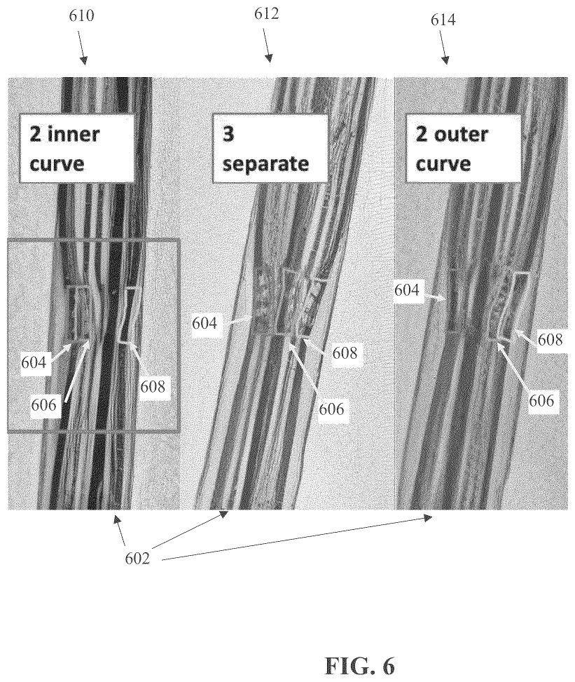

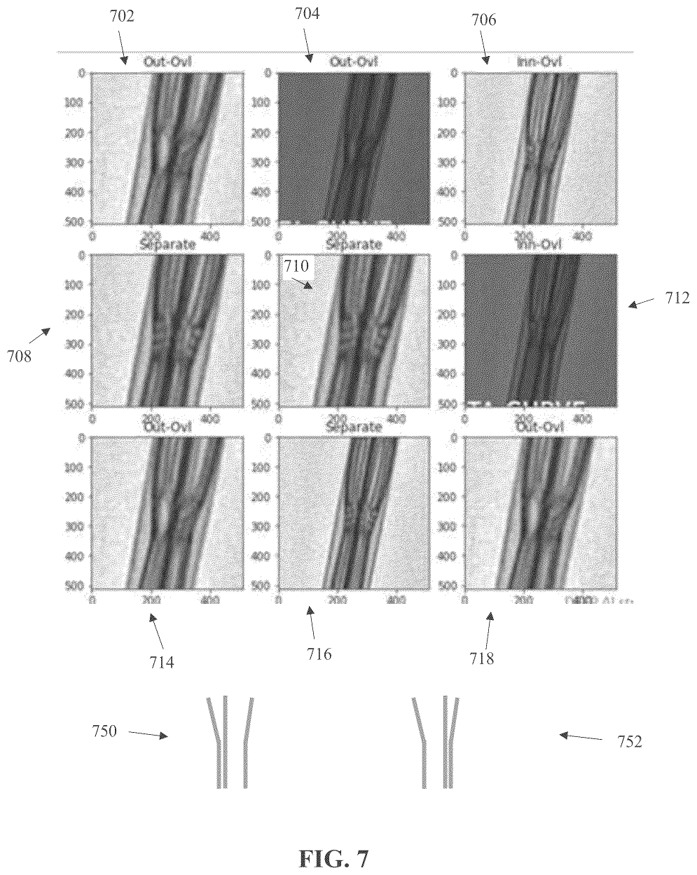

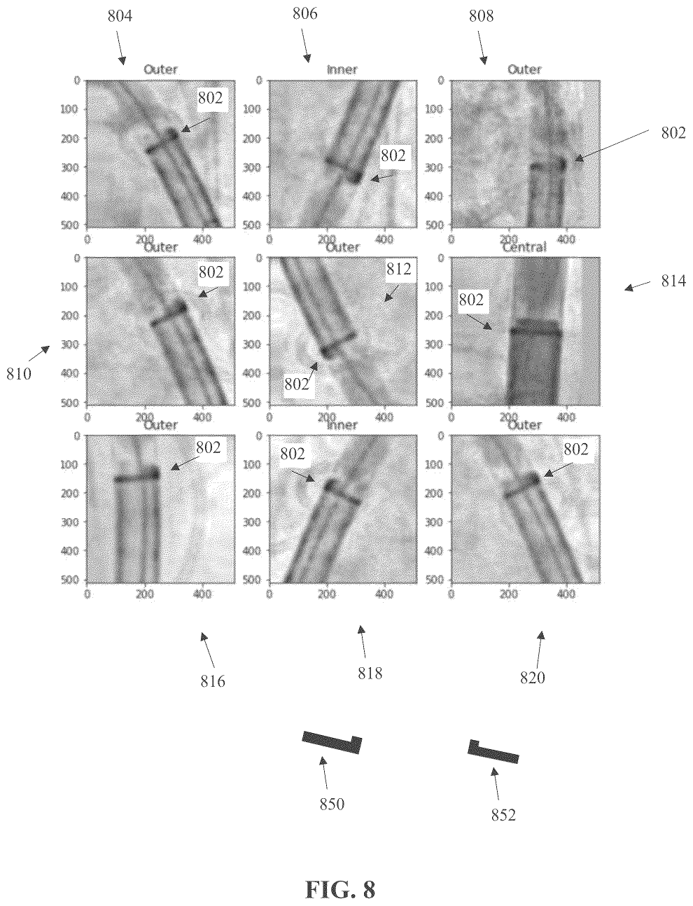

As used herein, the term roll angle is sometimes used as an example of an orientation. The term roll angle and orientation are sometimes used interchangeably. The roll angle may refer to rotation around a long (i.e., longitudinal) axis of the medical device. The orientation may include the roll angle, and/or other examples as described herein. For example, for a medical device, the roll angle may include, for example, whether the medical device is correctly oriented or not. For a prosthetic aortic valve, the roll angle may include, for example, whether the marker is aligned with the native commissure of the aortic annulus where the prosthetic aortic valve is to be deployed. The roll angle may not necessarily refer to specific angles, but rather to a classification category indication a range of rotations of the medical device and/or visual appearance of markers of the medical device that fall within a single category, for example, inner curve state, outer curve state, and middle state. In another example such as for a certain type of prosthetic aortic valve, the roll angle may refer, for example, to the example classification categories of inner-overlap, outer-overlap, and/or separate, described herein for example, with reference to FIG. 6 and/or FIG. 7. In yet another example such as for a different type of prosthetic aortic valve, the roll angle may refer to, for example, central, outer, and inner, described herein for example, with reference to FIG. 8.

As used herein, the terms orientation and orientation characteristic are used interchangeably. As used herein, the term first image, original image, target image, target original image, and initial image may sometimes be interchanged. The aforementioned terms may refer to the raw image captured by the medical imaging device, which is enhanced and/or analyzed, for example, the roll angle and/or marker are determined.

As used herein, the term sample image(s) may refer to the images used for training ML models. The term sample original medical image may refer to the raw image captured by the medical imaging device.

An aspect of some embodiments of the invention relates to systems, methods, computing devices, and/or code instructions (stored on a data storage device and executable by one or more processors) for generating a presentation of at least one image for assisting an operator in bringing a medical implement having a marker to a target location within a body of a patient. A processor(s) receives an initial image (also referred to herein as a first image) of the medical implement in the body of the patient, while the implement is on its way to the target location for example, an x-ray image depicting an aortic valve prosthesis in the aorta on the way to the aortic annulus. The processor(s) processes the initial image to obtain an enhanced image showing at least the marker. Optionally, the enhanced image is of a region of interest (ROI) showing the marker, which may include a portion of the aortic valve prosthesis in proximity to the marker and exclude a remainder of the aortic valve prosthesis. The processor(s) may feed the initial image into a ML model trained to identify the ROI encompassing the marker, and/or trained to generate the enhanced image depicting the ROI that includes the marker. The enhanced image is provided for display. Optionally, the enhanced image is provided for display as an inset on a display of the initial image.

An aspect of some embodiments of the invention relates to systems, methods, computing devices, and/or code instructions (stored on a data storage device and executable by one or more processors) for guiding a trans-catheter aortic valve replacement intervention in a patient.

An aspect of some embodiments of the invention relates to systems, methods, computing devices, and/or code instructions (stored on a data storage device and executable by one or more processors) for computing a state of a marker of a medical device for guiding a trans-catheter aortic valve replacement intervention in a patient, optionally an orientation of the medical device, optionally the orientation is of an aortic prosthetic valve, for example, indicating whether the aortic prosthetic valve is properly orientated for implantation or not, such as whether commissures of the aortic valve prosthesis are aligned with opening of the coronary arteries (coronary ostia) or not. A processor(s) obtains fluoroscopic images capturing the aortic valve prosthesis device in the aorta of the patient, for example, the aortic bulb, the aortic arch, the ascending aorta, and/or the descending aorta. The processor feeds at least one of the obtained images into to a machine-learning model trained to identify a state of a marker on the valve prosthesis device. The processor receives output indicating the state of the marker from the machine-learning model. The processor generates instructions for displaying the indication of the state of the marker according to the received output.

An aspect of some embodiments of the invention relates to systems, methods, computing devices, and/or code instructions (stored on a data storage device and executable by one or more processors) for training a ML model for determining an orientation of an aortic valve prosthesis for trans-catheter deployment (i.e., in the compressed state) depicted in a medical image. It is noted that the aortic valve prosthesis is an example, and the approach for training the ML model may be applied to other medical devices. For each sample medical image of multiple sample original medical images of subjects, where each sample original medical image depicts the aortic valve prosthesis with a marker: a region of interest (ROI) of the sample original medical image that depicts the marker is defined, for example, a frame. An enhanced medical image is created from the ROI, as described herein. An orientation of the aortic valve prosthesis depicted in the enhanced medical image is determined. The orientation may be based on the visual presentation of the marker in the enhanced image. Using the enhanced medical image may enable a more accurate determination of the orientation than using the original image. A record that includes the sample original medical image and a ground truth indicating the orientation, is created. A training dataset of multiple records is created. The ML model is trained on the training dataset for generating an outcome of the orientation in response to an input of a target original image depicting a target aortic valve prosthesis for trans-catheter deployment. Variations of the ML model are described herein, for example, the ML model may generate the enhanced image, and/or the ML model may include two components.

The orientation of the medical device may be determined according to the relative locations of one or more markers depicted in the 2D image of the medical device using embodiments described herein such as the ML model(s) and/or other image processing approaches, optionally while the medical device is in the compressed state. In some embodiments, the medical device is an aortic valve prosthesis and includes three markers spaced apart along a circumference thereof (e.g., circumference of the aortic valve prosthesis), for example, each one of the three markers is placed at a commissures of the prosthetic valve. In implementations in which the medical device includes three markers spaced apart along a circumference thereof (e.g., circumference of the aortic valve prosthesis) exemplary orientations based on the way the three markers are seen in the 2D image include: two markers overlapping on a left side of the image and a third marker does not overlap, two markers overlapping on a right side of the image and a third marker does not overlap, and none of the three markers are overlapping. In implementations in which the medical device includes a single main marker, the orientation may refer to the location of the single main marker, for example, outer (e.g.,--\hat{ }), inner;

which may be the mirror image of the outer orientation (e.g., \hat{ }--), and central (i.e., approximately in the middle, away from the sides, e.g.,-\hat{ }-). The orientation of the aortic prosthetic valve may be detected regardless of the orientation of the marker itself, for example, detecting the marker in different poses (e.g., rotations) may all correspond to outer. Alternatively or additionally, the orientation may be a binary classification, indicating whether the device is in a correct orientation or an incorrect orientation. When the device is the aortic valve prosthesis, correct orientation may indicate that the commissures of the prosthetic valve are non-aligned with the coronary ostia. Incorrect orientation may indicate that commissures of the prosthetic valve are aligned with the coronary ostia.

The examples of orientations may serve as classification categories for training the ML model and/or which are outputted by ML model(s).

Optionally, a target pose of an imaging sensor capturing a target fluoroscopic image for which the indication of the state of the marker (e.g., orientation of the aortic prosthetic valve) is obtained, is determined. The target pose may be the pose (e.g., selected by the operator, and/or selected automatically) at which the orientation of the aortic prosthetic valve may be determined from an image captured by an imaging sensor at the target pose, for example, the outcome of the ML model fed the image captured at the target pose is above a threshold indicating sufficient accuracy and/or probability of correctness. The operator may adjust the pose of the imaging sensor from the target pose to a new different pose. For example, the target pose is used to rotate the prosthetic aortic valve located in the descending aorta. The operator may then adjust the pose for advancing the prosthetic aortic valve through the aortic arch and/or into the ascending aorta. The orientation of the aortic prosthesis may not be determinable, and/or not accurately determinable using the markers depicted in images captured at the new pose. For example, the new pose may not clearly show the locations of the markers to enable the determining the orientation of the valve. For example, the ML model fed the image captured at the new pose may generate an inaccurate outcome, and/or may be below the threshold sufficient accuracy and/or probability of correctness. The new pose defines a new parallax, which may make the received x-ray signal different and/or weaker, making it difficult or impossible to see the marker(s) and/or determine the orientation according to the marker(s). The aforementioned technical problem may be addressed by computing a transformation function for transforming an image from the new pose to the target pose. The transformation function is applied to at least a portion of the second fluoroscopic image depicting the marker for obtaining a transformed image depicting the marker at the target pose. For example, a bounding box around the marker at a portion of the heart valve is computed, and the transformation function is applied to the bounding box. The transformed image may be presented on a display, optionally simultaneously with images captured at the new pose. The transformed image may be dynamically computed and updated in real time, as the operator captures new images at the new pose. The operator may refer to the transformed image to check that the markers indicate that the valve is oriented correctly, such as when the current images at the new pose are unsuitable for checking that the markers indicate that the valve is oriented correctly. For example, the operator uses the current images captured at the new pose to guide the valve over the aortic arch, while also referring to the transformed image to check that the valve is properly oriented. These embodiments may be combined with other embodiments, for example, the first image (or original image) referred to herein may be the current images captured at the new pose, while the enhanced image referred to herein may be the transformed image.

At least some embodiments described herein address the technical problem of determining an orientation of an aortic valve prosthesis on 2D images, optionally fluoroscopic images, for deployment of the aortic valve prosthesis (e.g., in the aortic annulus). At least some embodiments described herein improve the technical field of image processing, by determining an orientation of an aortic valve prosthesis on 2D images, optionally fluoroscopic images, for deployment of the aortic valve prosthesis (e.g., in the aortic annulus). The aortic valve prosthesis commonly includes three leaflets, secured at commissures. During a trans-catheter procedure for deploying the aortic valve prosthesis, the valve is to be oriented such that the commissure are not aligned with coronary ostia within the aortic bulb. Since the leaflets do not move at the commissures, alignment of the commissures with the coronary ostia blocks or reduces blood flow into the coronary arteries. Non-alignment of the commissures with the coronary ostia enables blood flow into the coronary arties, since the coronary ostia are not blocked by the leaflets. The orientation of the aortic valve prosthesis is commonly determined at the descending aorta, where rotation of the aortic valve prosthesis may be performed by the operator, for example, to obtain a target orientation. Once the aortic valve prosthesis has been advanced across the aortic arch, the aortic valve prosthesis is not commonly rotated, since rotation may cause the aortic valve prosthesis to apply a shear force to the wall of the aorta. To rotate the aortic valve prosthesis when it is located in the aortic arch or past it (e.g., in the ascending aorta), the aortic valve prosthesis is retracted back into the descending aorta, rotated, and then re-advanced. As such, accurate determination of the orientation of the aortic valve prosthesis in the descending aorta helps accurate deployment positioning, and/or reduces likelihood of risk to the patient and/or wasted time when the orientation is found to be incorrect when the valve prosthesis is in or past the aortic arch (e.g., ascending aorta, aortic annulus). Determination of the orientation of the aortic valve prosthesis in the descending aorta is difficult, since the aortic valve prosthesis is in the compressed state. For example, when the aortic valve prosthesis includes three markers located at the commissures, the orientation is difficult to determine on fluoroscopic images of the compressed aortic valve prosthesis in the aorta.

At least some of the methods, computing devices, code instructions, and/or systems described herein provide enhancement of images of the medical implement on its way to the target location inside the patient's body, to help the operator (e.g., surgeon) identifying the orientation of the medical implement, preferably, when the operator (e.g., surgeon) is still in control of that orientation. In some embodiments, the computing devices, code instructions, system, and/or methods includes explicitly indicating the orientation of the medical implement to the operator (e.g., surgeon), for example, by visual, textual, and/or or audio indication, presented on a display and/or played on speakers. When the operator (e.g., surgeon) identifies that the orientation of the implement has to be corrected, and optionally also what direction and extent of correction is required, the operator (e.g., surgeon) may take the initiative to correct the orientation of the implement according to the assistance provided by the methods and systems described herein.

At least some embodiments described herein address the technical problem of visualizing medical devices during a trans-catheter procedure, for example, during delivery of an aortic valve prosthesis for implantation in the aortic annulus, such as while the valve prosthesis is in the compressed state and/or located in the aorta. At least some embodiments described herein improve the field of medical image processing, and/or of machine learning models that process medical images, for example, 2D x-rays (also referred to as fluoroscopic images). Visualizing medical devices during the trans-catheter procedure is based on indirect visualization by the operator, using images captured of the medical device in the body, for example, x-rays. This is in contrast to standard surgical procedures where the surgeon opens up the body to expose the target location, and sees the target location and the medical device during implantation. While trans-catheter procedures are substantially less invasive than open heart surgery, the lack of line-of-sight visualization of the prosthetic heart valve and the native valve presents challenges, because the physician cannot see the actual orientation of the prosthetic heart valve during the implantation procedure. Correct positioning of the prosthetic heart valve is achieved using radiographic imaging (e.g.; fluoroscopic imaging), which yields a two-dimensional image of the viewed area. The physician must interpret the image correctly in order to properly place the prosthetic heart valve in the desired position. Failure to properly position the prosthetic heart valve sometimes leads to migration of the prosthetic heart valve or to improper functioning. Proper placement of the prosthetic heart valve using radiographic imaging is thus critical to the success of the implantation.

In one example, trans-catheter aortic valve replacement (TAVR) is a minimally invasive heart procedure to replace, for example, a thickened aortic valve that can't fully open, a condition known as aortic valve stenosis. The aortic valve is located between the left ventricle and the aorta. If the valve doesn't open correctly, blood flow from the heart to the body is reduced. TAVR can help restore blood flow and reduce the signs and symptoms of aortic valve stenosis-such as chest pain, shortness of breath, fainting and fatigue. Trans-catheter aortic valve replacement may also be called trans-catheter aortic valve implantation (TAVI).

Some embodiments described herein address the above mentioned technical problem, and/or improve the above mentioned technical field, by helping in carrying out a medical intervention that includes bringing a medical implement to the target location when the implement is properly oriented. In some embodiments, the implement includes a marker, configured to indicate the orientation of the implement. For example, the marker is made of radio-opaque material designed to be clearly visible on x-ray images. In some embodiments, the orientation in question is along the roll coordinate of the medical implement, that is, the orientation around a longitudinal axis of the medical implement, also referred to here as the roll angle. One example of such an intervention is a trans-catheter aortic valve replacement or implantation (also known as TAVR or TAVI). In one example, there may be multiple markers, positioned around a circumference of the medical device, such as the aortic valve prosthesis in a predefined pattern (e.g., preset arc spacing along the circumference and/or preset spacing along a long axis). The orientation, such as roll angle, may be determined according to an analysis of the pattern of markers depicted in the image(s), which may be 2D x-ray images.

At least some embodiments described herein relate to the technical problem of obtaining a target orientation of the medical device for implantation. At least some embodiments described herein improve the technical field of medical processing, by analyzing 2D images (e.g., x-ray) for determining the target orientation of the medical device for implantation. In TAVI, it is important to align commissures of the medical implement with the commissures of the native aortic valve. Commissural malalignment may lead to varying degrees of overlap between the neo-commissural posts and coronary arteries. Furthermore, experimental models have shown that trans-catheter heart valve leaflet stress and central aortic regurgitation (AR) may be exacerbated with suboptimal commissural alignment. These findings have significant clinical implications for younger patients who have an increased lifetime risk of complications of aortic valve disease and coronary artery disease.

Some medical devices and/or other aortic valve prosthetic devices are to be oriented in alignment with the native commissure of the native heart valve, may include markers that may be used for helping the operating surgeon implanting the prosthetic device at the correct orientation. In some of the medical devices the marker include multiple of marking elements, e.g., radiopaque dots or short lines, that are oriented one in respect to the other in some predetermined manner when, and only when, the device is properly oriented.

However, the markers are frequently hard to find in the image, and the operating surgeon is required to invest considerable cognitive resources for finding it in the image, and determining the state of the marker, which is indicative to the orientation being proper or not.

It is noted that the appearance of the marker in the 2D image depends not only on the way the marker is aligned in respect to the patient, but also on the positioning of the imager. Therefore, it is not necessarily sufficient to identify how the marker appears, but also the viewing angle at the image was taken. Usually, the implantation of the prosthetic valve device is carried out in a certain, preferable, fluoroscopic view (i.e., “cusp overlap view”).

At least some embodiments described herein address the aforementioned technical problem, and/or improve the aforementioned technical field, by supplying the operator (e.g., surgeon) with adequate information on the orientation of the medical implement, thus helping the operator (e.g., surgeon) in orienting the heart valve implement in the aorta to reduce the probability of commissural malalignment.

At least some embodiments described herein address the aforementioned technical problem, and/or improve the aforementioned technical field, by identifying the appearance of the marker, and optionally also the fluoroscopic view, (e.g., for the surgeon). At least some embodiments described herein provide apparatuses and/or computer implemented methods for determining the orientation of the prosthetic device from fluoroscopic images.

As used herein, guiding an intervention (e.g., trans-catheter) may include providing information about the manner at which the intervention proceeds. For example, the information may include images of the interior of the patient's body, where intervention takes place and/or where a medical device operated by the operator (e.g., operating surgeon) is navigating. The information may be provided to the operator (e.g., operating surgeon) or to any other member of the operating stuff, by being displayed on a display.

At least some embodiments described herein address the aforementioned technical problem, and/or improve the aforementioned technical field, by identifying the appearance of the marker, and optionally the fluoroscopic view. At least some embodiments described herein provide apparatuses/or and computer implemented methods for determining the orientation of the medical device (e.g., prosthetic device) from fluoroscopic images.

An aspect of some embodiments of the invention relates to systems, methods, computing devices, and/or code instructions (stored on a data storage device and executable by one or more processors) for a processor(s) receiving an image of the medical implement on its way to the target location in the body of the patient. This image is sometimes referred to herein as a first image or an original image or an initial image. Optionally, the original image is a fluoroscopic image, optionally 2D, but the technology is not limited to fluoroscopy, and other images may be used, if available, for example, ultrasound images. The original image shows the medical implement, optionally in real time or near real time, during the medical intervention.

The processor(s) may further process the original image to obtain an enhanced image of at least a portion of the medical implement in the body of the patient. The portion shown in the image preferably encompasses the orientation marker. The enhanced image may be of a higher quality than the original image, for example, improved visual depiction of the at least the portion of the medical implement and/or improved visual depiction of the marker and/or improved visual depiction of the anatomical structures in close proximity to the medical implement. The image processing may include one or more image enhancement techniques, for example, filtering with morphological operators; histogram equalization; noise removal (e.g., using a Wiener filter), linear contrast adjustment; median filtering; unsharp mask filtering, contrast-limited adaptive histogram equalization, and/or decorrelation stretch. Alternatively or additionally, the image processing of the original image to obtain the enhanced image is performed by feeding the original image into a ML model trained to generate the enhanced image, for example, a neural network, which may be a generative neural network, for example, part of a generative-adversarial network (GAN).

Then, both the original image and the enhanced image are provided by the processor for display on a display, for example, to the surgeon. The original image provides the overall context of the current stage in the intervention, showing the full field of view of the imager that took the image (e.g., the fluoroscope). The enhanced image may include a small portion of the field of view of the original image and/or may exclude regions of the original image external to the small portion. The portion depicted in the enhanced image includes the medical implement or at least a portion of the medical implement that includes an orientation marker. The enhanced image may be of smaller field of view than the original image, and optionally also of smaller dimension. In some embodiments, the enhanced image is enlarged, so that the marker is easier to see not only because of the enhanced image quality, but also thanks to the enlargement of the image. Optionally, the enhanced image is shown as an inset on the original image presented on a display and/or within a graphical user interface (GUI).

The generation of the presentation of the inset of the enhanced image on the original image may, for example, be beneficial to the surgeon who can easily turn attention from the general context provided by the original image to the specific context of the orientation marker, the view of which is enhanced in the inset. Using an inset may further obviate the need for registration between the enhanced image and the original one. In some embodiments, an inset is also advantageous over an enhanced image of the entire field of view shown in the original image, because enhancing the entire image might improve the clarity of many details irrelevant to the orientation, and thus drown the orientation marker in a sea of less important details.

Thus, in some embodiments, the processing includes identifying a portion of the original image as a region of interest comprising the marker, cropping the identified portion from the first image, and enhancing the cropped portion of the first image (e.g., enhancing only the cropped portion). Optionally, the entire image is enhanced, and then a portion thereof is cropped, optionally enlarged, and provided for display. Alternatively or additionally, the original image is fed into a ML model trained to generate an outcome of the enhanced image which includes the cropped portion of the first image.

Optionally, both the original image and the enhanced image are displayed on the same display device, for example, on the display device of the imager. Alternatively or additionally, the original image and the enhanced image are presented with a GUI that provides other features, for example, presentation of an indication of orientation of the medical device according to the marker.

In some embodiments, the identification of the region of interest may be carried out using a machine-learning model trained to identify the region of interest. Such a machine-learning model may be trained on a training set of images, where the region of interest was marked manually and/or using a non-supervised approached where the region of interest was learned automatically. For example, a frame of predetermined dimensions may be provided to a surgeon, and the surgeon may put the frame around the part of an (original) image that is most informative to him. In another example, the frame of predetermined dimensions may be provided to the ML model as a learning parameter, and the ML model learns automatically how to fit the frame. The frame may be placed such that the marker is at about the center of the frame. The training set may include many different images with frames and/or from which placement of the frame is learned. The images in the training set may include images taken during procedures of different patients optionally of a same type (e.g., TAVI) optionally of a same type of medical device (e.g., same type of aortic valve prosthesis), and optionally from each procedure, many images may be included.

The training set may include images received from the imager. Individual images may be transformed, optionally randomly, e.g., by rotation, zoom, and/or shift. In some embodiments, each individual image may be transformed for example, up to about 10, or 50, or 100 additional times (or other values), so for example, with 300 original images it is possible to generate a training set of 30 000 slightly different images, which may decrease the risk of overfitting. This may enable the machine-learning model to be trained on many more images than those collected and labeled. The amount of the images used in the training may be increased without having to collect and label addition images. Optionally, the training is done using stochastic optimization, and each epoch a different set of the images is used in the training.

The training set may further include images in which the implement and/or marker is not shown, so as to train the model not to identify regions of interest in images that don't include such region with marker. Such images without the implement and/or without the marker may lack the frame, and/or be labelled with a tag that indicates that the image does not depict the implement and/or the marker.

Optionally, the training is done using stochastic optimization, and a different sub-set of the training-set is used for each epoch in the training.

ML models described herein may be implemented, for example, as one or combination of: a classifier, a statistical classifier, one or more neural networks of various architectures (e.g., convolutional, fully connected, deep, encoder-decoder, recurrent, graph, combination of multiple architectures, GAN), support vector machines (SVM), logistic regression, k-nearest neighbor, decision trees, boosting, random forest, a regressor and the like. ML model(s) may be trained using supervised approaches and/or unsupervised approaches on training dataset(s), for example, as described herein.

In some embodiments, further to displaying an enhanced image of the marker containing portion of the medical implement, the processor may further estimate an orientation (e.g., roll angle) of the medical implement, and may provide for display an indication to the estimated orientation. The roll angle (or other orientation characteristic) may be estimated based on the way the marker appears in the image. For example, the roll angle may be estimated based on the size of the marker, the position of the marker in relation to other parts of the medical implement, the shape of the marker, and/or the way a pattern of markers with a predefined distribution on the medical implement appear in the original image and/or in the enhanced image. Alternatively or additionally, the roll angle (and/or other orientation characteristic(s)) is obtained as an outcome of the ML model in response to feeding the original image and/or the enhanced medical image into the ML model. Alternatively, there may be two ML model components. A first ML model component generates the enhanced image in response to being fed the original image. A second ML model component is fed the outcome of the first ML model, i.e., the enhanced image, and generates the orientation as an outcome. Alternatively, there may be three ML model components, where the original image is fed into the first ML model, the outcome of the first ML model is fed into the second ML model, the outcome of the second ML model is fed into the third ML model, and the outcome of the orientation is obtained from the third ML model, as described herein.

The marker may be sized and/or shaped for appearing in the image with characteristics that are indicative to the roll angle. For example, in some embodiments, the marker is shaped to display on the image a portion of its length depending on the roll angle. In another example, some markers are made of two or more marking elements, and the roll angle is indicated by the spatial relationships between them.

In some embodiments that include explicitly indicating the orientation of the medical implement, e.g., by visual, textual, or audio indication, the processing of the original image may include inputting the original image to the machine-learning model trained to estimate the roll angle.

Optionally, the training set includes training images (e.g., as described herein), with ground truth labels indicating the orientation and/or orientation state of the implement. Exemplary ground truth labels include: whether the orientation is proper or not, and/or the amount of orientation, for example, in degrees. Another option is to label the enhanced images, rather than the original ones. The latter option of labeling the enhanced images may be easier, because identifying the orientation in the enhanced images may be easier. This option may be carried out by first labeling the original images with the regions of interest, then enhancing image portions comprising the regions of interest, and then using the enhanced images for labeling these images (and/or the respective original images) with orientation labels.

Optionally, a display of a cine of enhanced images is created. For example, the imager can be operated continuously and/or at predefined intervals, for generating a presentation on a screen of a cine of the scene made of original images, and on the screen displaying the cine, there is another (optionally inset) cine, of the enhanced images. This may be useful to ensure that whenever the surgeon looks at the inset, the inset reflects the current situation. This may require repeating the generation of the enhanced image at least 10 times a second, or any higher rate that allows generating a cine that appears continuous and not flickering for the human eye.

An aspect of some embodiments of the present invention is a system configured to carry out a method as described herein. Such system includes a memory storing instructions, and a processor, configured to execute the instructions. The system may also include an input for receiving the original images, and an output to a display device. The display device may be the display of the imager. The instructions saved on the memory include instructions for the processor regarding receiving the original images via the input and providing the processing results via the output.

Optionally, methods described herein are implemented on a computer, fully automatically and/or mostly automatically (e.g., some labelling of images for training the ML model may be done manually). The computer includes a processor(s) and a digital memory that stores instructions, that when executed by the processor cause the processor to carry out one or more of the methods described herein. In some embodiments, the computer may also include a display for displaying information for guiding the intervention.

At least some implementations of systems, methods, and/or code instructions described herein do not automate manual tasks in the same way they had been previously carried out (e.g., the operator mentally determines the orientation of the medical device based on visual inspection of the marker on x-rays), but create a new automated process based on images, where the new automated process includes (alone or in combination) new features that have never been performed before and/or features that have no manual counterpart, for example, automated enhancement of ROIs extracted from original images, training of ML models, inference by ML models, and/or other features described herein.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

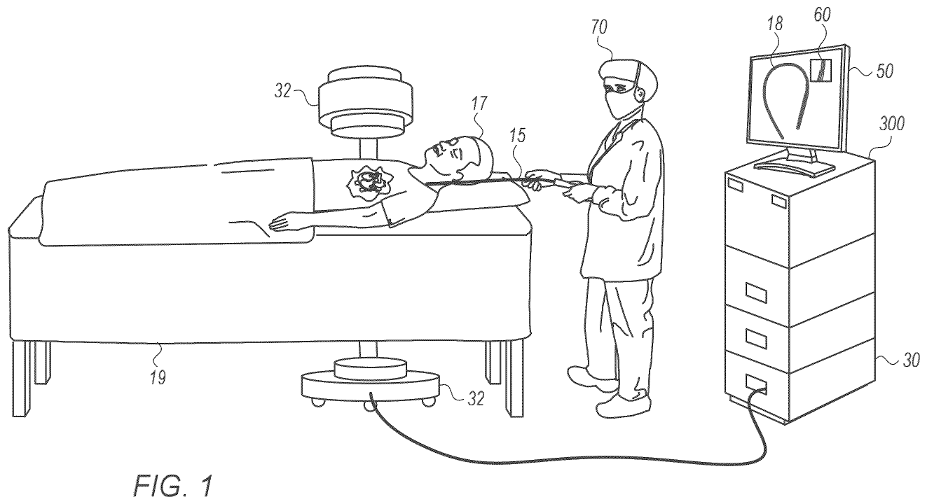

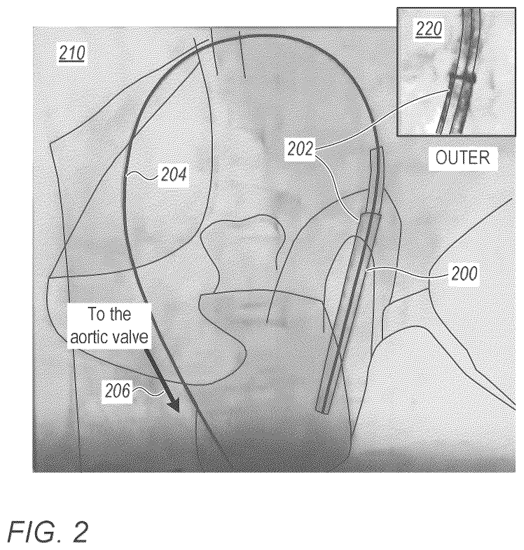

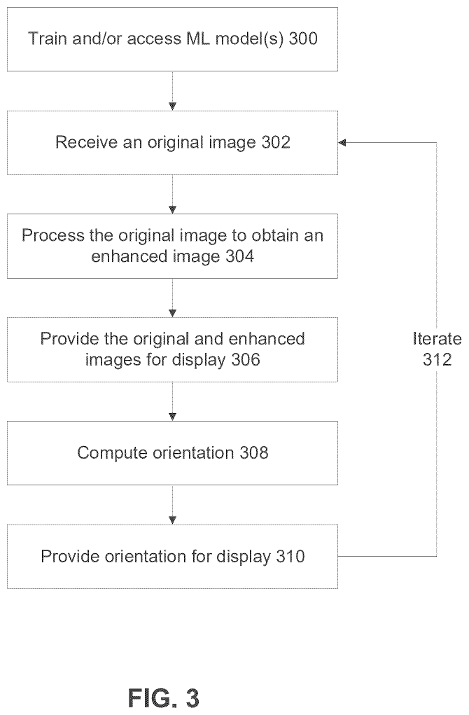

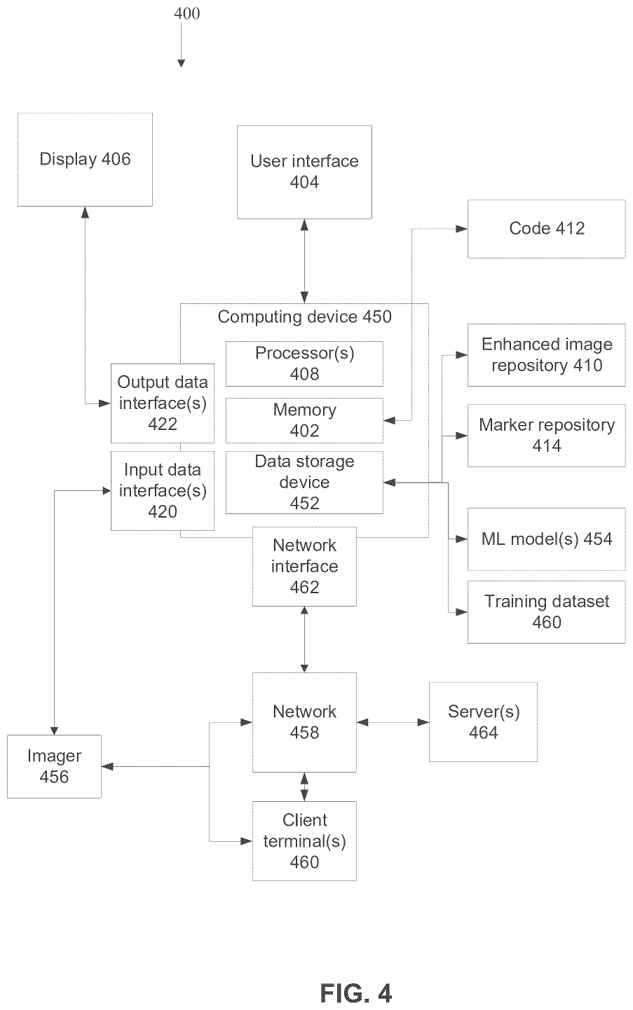

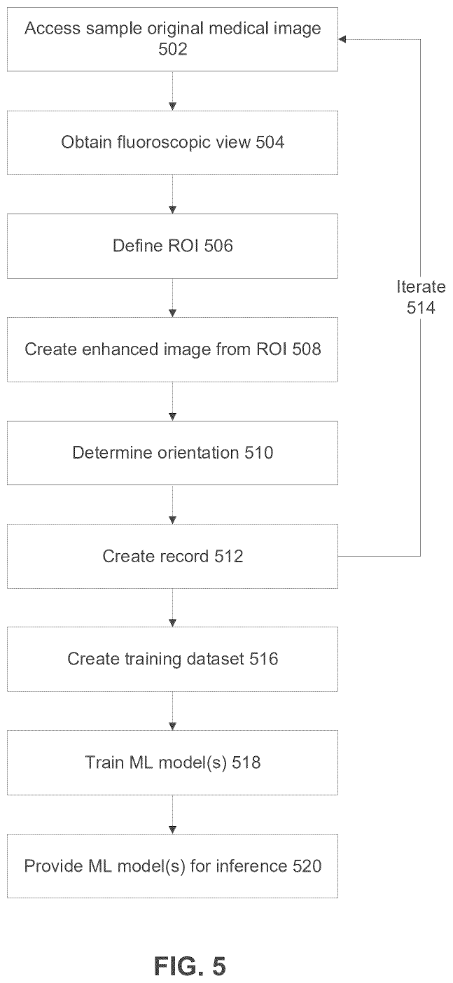

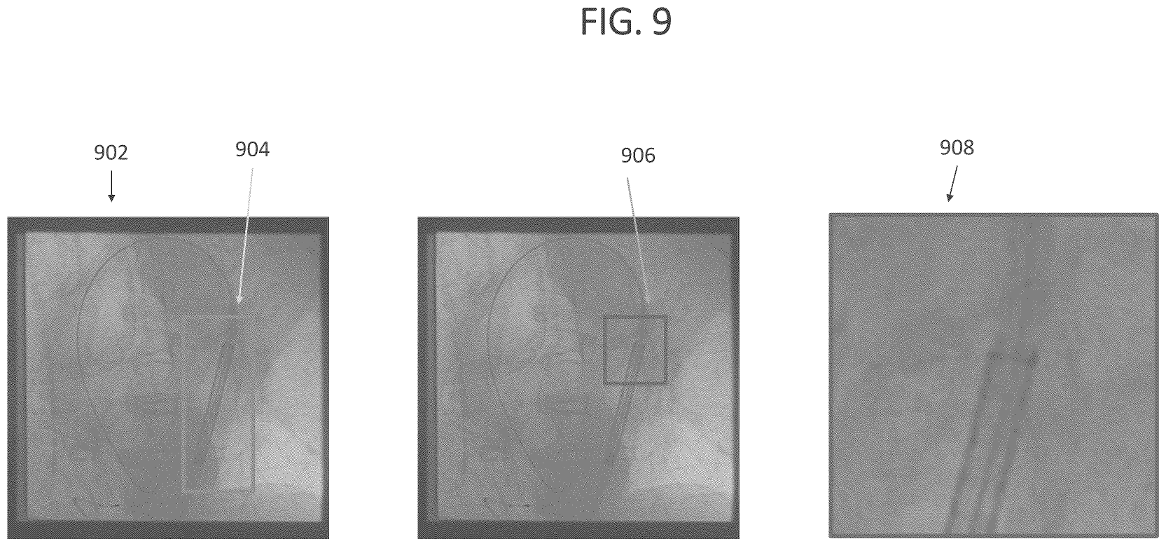

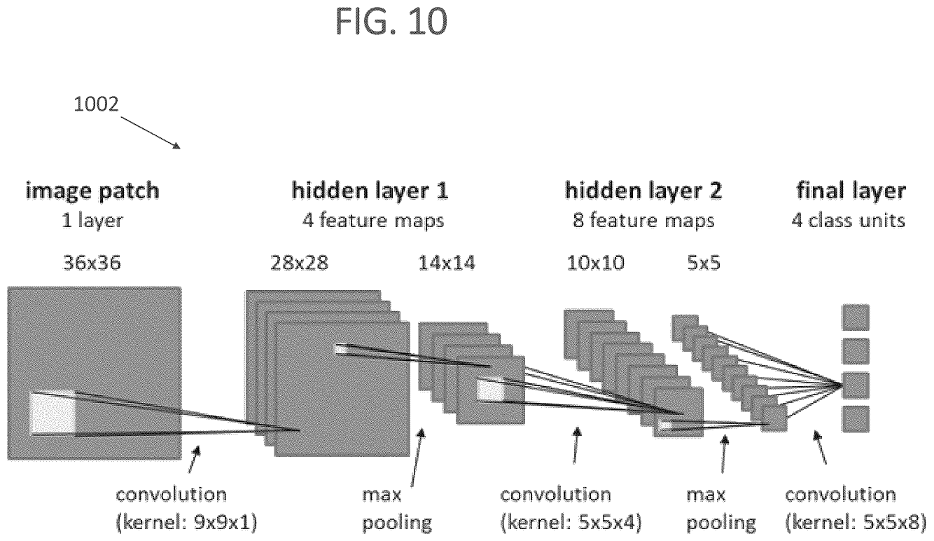

Reference is now made to FIG. 1, which is a pictorial illustration of an operating room equipped with a system for guiding medical procedure, in accordance with some embodiments of the present invention. Reference is also made to FIG. 2, is an exemplary display of an original fluoroscopic image 210 displayed with an enhanced image 220 as an inset, in accordance with some embodiments of the present invention. Reference is also made to FIG. 3, which is a simplified flowchart of a method for generating a presentation for assisting an operator in bringing a medical implement to a target location within a body of a patient, according to some embodiments of the invention. Reference is also made to FIG. 4, which is a simplified block diagram of a system configured to carry out the method of FIG. 3 and/or FIG. 5, in accordance with some embodiments of the present invention. Reference is also made to FIG. 5, which is a simplified flowchart of a method of training ML model(s) for generating an outcome of an orientation of a medical device and/or an enhanced image, in accordance with some embodiments of the present invention. Reference is also made to FIG. 6, which is a schematic depicting exemplary orientations of an aortic valve prosthesis with three spaced apart markings, in accordance with some embodiments of the present invention. Reference is also made to FIG. 7, which includes schematics of different orientations of an aortic valve prosthesis that includes three markers located at the commissures of the aortic valve prosthesis, in accordance with some embodiments of the present invention. Reference is also made to FIG. 8, which includes schematics of different orientations of an aortic valve prosthesis that includes a single main marker, in accordance with some embodiments of the present invention. Reference is also made to FIG. 9, which is a schematic depicting a sample original image 902, a first boundary box 904, a second boundary box 906, and an enhanced image 908 which is labelled with the ground truth of orientation of the valve prosthesis according to the depicted marker, in accordance with some embodiments of the present invention. Reference is also made to FIG. 10, which is a schematic 1002 of an exemplary neural network architecture of ML model(s), in accordance with some embodiments of the present invention.

Referring now back to FIG. 1, system 300 is depicted as being used for guiding a trans-catheter procedure, for example, a TAVI intervention, or other procedure in which it is advantageous to help the surgeon controlling the orientation of a medical implement, optionally by determining the roll angle of the medical implement. System 300 may be used for guiding any medical procedure in which the orientation of an intrabody medical tool, including one or more markers, is required. A catheter 15 is percutaneously inserted into a living body 17 of a patient lying on a gurney 19. Catheter 15 is controlled and manipulated by operator 70 (e.g., a surgeon). An imaging system 30 (sometimes referred to herein as an imager), is used to obtain an image of the inside of the body of the patient for guiding the medical implement. For example, an image of the aorta in which the medical implement is located. Imaging system 30 is shown to include an imaging source 32 (sometimes referred to herein as an imager), which may use, for example, magnetic resonance imaging (MRI), X-ray computed tomography (CT), fluoroscopy (i.e., 2D x-rays) and/or any suitable imaging technique to obtain the image(s) of the interior of the body, such as of the aorta. The image (e.g., of the aorta) may be digitized and/or sent to system 300 for processing.

An image 18 (e.g., a fluoroscopic image of the aorta) is displayed to operator 70 on an output display 50, and/or a copy of the image may be sent to system 300 for processing and/or generating an enhanced image 60 shown with the fluoroscopic image and/or for analysis for determining the orientation of the medical implement. The processing may be carried out fast enough so that any time delay between the original image and the enhanced image is too short to be felt by the human eye. In other words, the processing may be fast enough so that the original image and the inset are practically synchronous with each other.

Referring now back to FIG. 2 and FIG. 3, at block 300, one or more ML models are trained and/or accessed, as described herein. An exemplary approach for training ML model(s) is described, for example, with reference to FIG. 5.

In block 302, an image of the medical implement in the body of the patient is received. The image is optionally received in real time from the imager, e.g., fluoroscope 32. An original image 210 of Evolut™ aortic valve implant in the ascending aorta, together with inset of enhanced image 220, is shown in FIG. 2. The Evolut™ aortic valve implant (i.e., medical implement) 200, which is shown in the compressed state (i.e., for delivery within blood vessels to the aortic annulus, has a radiopaque marker 202, shown both in original image 210 and in enhanced image 220. Aortic valve implant 200 moves on guidewire 204 up towards the aortic arch, and from there through the ascending aorta towards the aortic valve (not in the image) in the direction of arrow 206.

The image may be a fluoroscopic 2D images capturing an aortic valve prosthetic device in the aorta of the patient, for example, the aortic bulb, the aortic arch, the ascending aorta, and/or the descending aorta. The prosthetic device may be, for example, Evolute™ by Medtronic, Porticco™ by Abbott, or LOTUS Edge™ by Boston Scientific.

The fluoroscopic images may be obtained from an imager that is integral to the guiding device, and/or from an independent imager. In some embodiments, the system (and/or computing device of the system) is directly connected to the imaging device, and receives as input data from the imager. Optionally, these data are the same used by the imager for producing and/or displaying the fluoroscopic image on the imager display. In some embodiments, the computer may include (or may receive input from) a camera that photographs the imaging device display, and the 2D images are obtained from this camera for purpose of the guiding. Such architecture may enable generating the enhanced image and/or computing the orientation using an external system that does not require setting up a connection for obtaining the 2D image (e.g., fluoroscopic image). In some embodiments, the fluoroscopic view is identified and read from an image of the imaging device display (e.g., using optical character recognition, and/or accessing metadata), and the computer may indicate this on its own display, and possibly refuse to provide feedback on the orientation and/or appearance of the marker if the view is not the preferable one.

The 2D images (e.g., fluoroscopic images) may be obtained and/or processed online, as described herein, for example, for generation of cine presented on a display. The real time processing may enabling the operating surgeon receiving the guidance in real time, although in some embodiments, the guiding may be provided after the fact, for post hock analysis and/or staff education.

In block 304, the original image (e.g., 50, 210) is processed to obtain an enhanced image (e.g., 60, 220) showing at least the marker (202) and optionally at least a portion of the aortic valve implant. Enhanced image 220 may be of a higher quality than original image 210. In the depicted embodiment, marker 202 is shown in enhanced image 220 more clearly than in original image 210. Additionally, enhanced image 220 is enlarged in comparison to the size of the same scene in original image 210.

The processing optionally includes identifying as a region of interest a portion of the original image that includes the marker. This identification may involve inputting the original image into a machine-learning model trained to identify the region of interest. The machine-learning model may be trained using a training set as described above. In other embodiments, the identification may include image processing methods to identify the region of interest.

The processing may further include cropping from the original image a smaller image, showing the region identified as the region of interest, and enhancing this portion. Other regions external to the region of interest may be excluded. In some embodiments, the entire original image may be enhanced, and the region of interest cropped from the enhanced image. The enhanced image may include an enlarged view of the region of interest, so the marker appears larger than in the original image.

Alternatively or additionally, the processing may include computing the transformed image from the original image, as described herein. The enhanced image may include and/or may refer to the transformed image.

In block 306, the original image and the enhanced image are delivered for presentation on a display, for example, to the surgeon. Optionally, the enhanced image is provided for display as an inset on a display of the first image presented on a screen, as shown in FIG. 1 and FIG. 2.

Optionally, in block 308, the orientation (e.g., roll angle) of the medical implement is estimated.

The orientation may be estimated based on the appearance of the marker in the enhanced image and/or in the original image. As identifying details of the marker appearance may be easier in the enhanced image, the enhanced image may be used as basis for estimating the orientation. Estimating the orientation may be performed, for example, by inputting the image (enhanced and/or original) to the machine-learning model described herein that is trained to identify the orientation from an inputted image. Providing the orientation for display (as in block 310) may include communicating with display device 50 via output interface, to control the display device to display the orientation, e.g., as a symbolic and/or textual indication of the estimated orientation.

Optionally, at least one of the obtained fluoroscopic images is fed to the machine-learning model trained to identify the orientation of a marker on the valve prosthetic device and output an indication to that orientation. For example, in some prosthetic devices, the marker may be in any one of four states: outer curve, inner curve, middle front, or middle back. The appearance of the marker at the latter two states is not distinguishable, so the machine-learning model may distinguish only between inner curve state, outer curve state, and middle state. The number of the available (distinguishable) states may depend on the kind of prosthetic device used, and it is usually between two and four.

The output from the machine-learning model may include, for example, indication to the appearance of the marker, and/or a binary output indicating whether the prosthetic device is properly oriented or not, and/or the state of the marker. The output may be received by a computer that controls a display to indicate the appearance of the device, and optionally, whether the device is properly oriented or not. In some embodiments, the display may be visual. e.g., a textual message may appear on the display, or an indicator may be lighted with lights of different colors, etc.

In some embodiments, the processor may generate an indication of the fluoroscopic view (also referred to herein as pose) or even, in some embodiments, if the orientation is proper or not, considering the fluoroscopic view and the marker appearance. The pose of the imager (e.g., fluoroscopic view) may refer to the view angle of the image sensor relative to the body of the patients. Examples of views include LAO, ROA, and others. The pose may be detected using optical character recognition (OCR) applied to the image when the image pose (e.g., LAO, RAO, etc. . . ) is indicated in the fluoroscopic image. In some embodiments, if the view is identified not to be the preferable (e.g., cusp overlap) view, the methods or apparatuses alert that the view is not the preferable one. In some embodiments, an indication if the orientation of the prosthetic device is proper or not may also be provided even if the image is obtained with an imager at non-preferable position.

In some embodiments, the training set for training the machine-learning model for estimating the orientation includes enhanced images of regions of interest. Optionally, the regions of interest are identified manually (e.g., as described above), cropped from the original image, enhanced, and provided for manual labeling according to the orientation of the implement, as identified by a human expert from the appearance of the marker in the image. Alternatively, or additionally, enhanced images of regions of interest identified by a trained machine-learning model are provided to a human expert for manual labeling according to the orientation of the implement.

In some embodiments, the training is with images labeled by human experts. The labeling may include a label of the orientation of the medical device, and/or if the medical device is properly or improperly oriented. In some embodiments, the machine-learning includes two modules, one trained to identify the prosthetic device in the image, and another trained to identify the appearance of the marker.

Optionally, in block 310, the estimated orientation is provided for display on a screen, optionally on the screen presenting the original and enhanced images (e.g., display 50).

At 312, one or more features described with reference to 302-310 may be iterated. The iterations may be done over a time interval, for each of multiple captured images. The images may be sequentially analyzed, and/or images may be sampled at a certain frame rate, which may be slower than the rate of captured, for example, such that the rate of images used is approximately the processing capability of the images using available computational resources.

In some embodiments, original images are received, processed, and provided for display at a rate of 10 times or more per second, 20 times or more per second, or at any rate that is high enough so that presenting the enhanced images sequentially generates a continuous (and preferably not flickering) cine of the region of interest, including the marker. In such embodiments, the operator (e.g., surgeon) can see at any time a current image of the region of interest, and even watch as the orientation changes, inadvertently, or in response to his control. In some embodiments, each iteration is of a new image obtained from the imager as the operation proceeds.

Optionally, from time to time (e.g., every 0.2, 0.5, 1, 2 or 5 seconds), a new image may be fed into the machine-learning model to check if the status of the marker has changed, for example, to help the operating surgeon realize the orientation of the device has changed, and if so, in what direction. In embodiments where an indication to the appropriateness of the orientation is provided such as presented on the display, the indications regarding changes in the marker state may indicate for example, if the operating surgeon succeeded in improving the orientation of the device in response to earlier provided feedback that the device is improperly oriented, or that the orientation, for some reason, changed for the worse, and the status changed from properly to improperly oriented.

In some embodiments, indication to a status change may include (optionally in addition to the change in the indication to the status itself) an audio display indicating the change, for example, a click or dong having different sounds to indicate the different status changes of the prosthetic device, e.g., improvement or setback.

Referring now back to FIG. 4, the block diagram of system 400 for generating a presentation for assisting an operator (e.g., surgeon) in bringing a medical implement (or any intra-body device) to a target location within a body of a patient, and/or for training one or more ML models, is depicted.

The embodiment depicted includes one or more of: a memory 402, storing code instructions 412; a processor(s) 408, configured to execute code instructions 412; an input data interface 420, for receiving original images from imager 456; and output data interface 422, for providing enhanced images to display 406, which in some embodiments is a display integral with imager 456. Additional exemplary components of system 400 are described herein.

The system is characterized by code instructions 412 that cause processor 408 to receive an original image (e.g., 210); process the original image to obtain an enhanced image (e.g., 220); and provide the original image and the enhanced image for display on display 406. Alternatively or additionally, the image is analyzed to detect a state of a marker on a medical implement (e.g., prosthetic heart valve), as described herein. In some embodiments, the enhanced image is provided for display as an inset on a display of the original image. Additionally, or alternatively, the processing includes applying to the original image a machine-learning model 454 trained to identify the region of interest. The region of interest includes a marker, which optionally indicates in real time the orientation (e.g., the roll angle) of the medical implement.

System 400 may implement the features of the method described with reference to FIG. 3, by one or more processors 408 of a computing device 450 executing code instructions 412 stored on memory 402 (also referred to as a program store).

Computing device 450 may receive images from imager 456, for example, directly over a network 458, and/or via a client terminal 460 in local communication with imager 456 (e.g., catheterization laboratory workstation), and/or via input data interface(s) 420 which may be a direct connection, and/or via a server 464 (e.g., PACS server).

Examples of imager 456 include a fluoroscopy and/or x-ray machine, for example, designed to be used during a percutaneous procedure, such as TAVI.

Multiple architectures of system 400 based on computing device 450 may be implemented, for example:

In an exemplary centralized architecture, computing device 450 may be implemented as one or more servers (e.g., network server, web server, a computing cloud, a virtual server) that provides services to one or multiple locations, for example, multiple catheterization laboratories and/or operating rooms, and/or multiple clinics. In such architecture, computing device 450 may receive images from imager(s) 456 located in different rooms for monitoring different ongoing catheterization procedures, as described herein. Computing device 450 may centrally provide image analysis services during the ongoing catheterization procedure to each of the rooms. Computing device 450 may be in communication with different client terminals 460 each located in a different room, for presenting generated images (e.g., enhanced images, and/or indication of state of the marker) on local displays and/or receiving input from different user interfaces used by different users.

In another example, computing device 450 may be implemented as an exemplary localized architecture, for example, for locally generating enhanced images and/or detecting a state of a marker for an ongoing catheterization procedure in a certain operating room and/or clinic. Computing device 450 may be implemented as, for example, code running on a local workstation (e.g., catheterization laboratory control station, surgical control station), and/or code running on an external device (e.g., mobile device, laptop, desktop, smartphone, tablet, and the like). Computing device 450 may be in local communication with imager(s) 456. Computing device 450 may be locally connected to imager(s) 456, for example, by a cable (e.g., USB) and/or short-range wireless connection and/or via network 458. In the localized implementation, the local computing device 450 may locally analyze the recordings from local imager(s) 456, and locally generate the enhanced image and/or determine the state of the marker. Computing device 456 may be installed, for example, in an operating room, ER, ambulances, Cath lab, or any other space in which a catheterization procedure is taking place.

Computing device 450 may be implemented as, for example, a client terminal, a server, a virtual machine, a virtual server, a computing cloud, a single computer, a group of connected computers, a mobile device, a desktop computer, a thin client, a Smartphone, a Tablet computer, a laptop computer, a wearable computer, glasses computer, and a watch computer.

Processor(s) 408 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 408 may include one or more processors (homogenous or heterogeneous), which may be arranged for parallel processing, as clusters and/or as one or more multi core processing units.

Memory 402 may be a digital memory that stores code instructions executable by hardware processor(s) 408. Exemplary memories 402 include a random-access memory (RAM), read-only memory (ROM), a storage device, non-volatile memory, magnetic media, semiconductor memory devices, hard drive, removable storage, and optical media (e.g., DVD, CD-ROM). Memory 402 may store code instructions 412 which implement one or more features (e.g., of methods) described herein.

Computing device 450 may include a data storage device 452 for storing data, for example, enhanced image repository 410 for storing the generated enhanced images, marker repository 414 for storing the determined state of markers, trained ML model(s) 454 and/or training dataset 460 for training ML model(s) 454. Data storage device 452 may be implemented as, for example, a memory, a local hard-drive, a removable storage device, an optical disk, a storage device, and/or as a remote server and/or computing cloud (e.g., accessed over network 458). It is noted that code may be stored in data storage device 452, with executing portions loaded into memory 402 for execution by processor(s) 408.

Machine-learning model(s) 454 may be implemented, for example, as one or combination of: a classifier, a statistical classifier, one or more neural networks of various architectures (e.g., convolutional, fully connected, deep, encoder-decoder, recurrent, graph, combination of multiple architectures), support vector machines (SVM), logistic regression, k-nearest neighbor, decision trees, boosting, random forest, a regressor and the like. ML model(s) 454 may be trained using supervised approaches and/or unsupervised approaches on training dataset(s), for example, as described herein.

Data interface(s) 420 and/or 422 may be implemented as, for example, one or more of, a wire connection (e.g., physical port), a wireless connection (e.g., antenna), a network interface card, a wireless interface to connect to a wireless network, a physical interface for connecting to a cable for network connectivity, and/or virtual interfaces (e.g., software interface, application programming interface (API), software development kit (SDK), virtual network connection, a virtual interface implemented in software, network communication software providing higher layers of network connectivity).

Computing device 450 may include a network interface 462 for connecting to network 458, for example, one or more of, a wire connection (e.g., physical port), a wireless connection (e.g., antenna), a network interface card, a wireless interface to connect to a wireless network, a physical interface for connecting to a cable for network connectivity, and/or virtual interfaces (e.g., software interface, application programming interface (API), software development kit (SDK), virtual network connection, a virtual interface implemented in software, network communication software providing higher layers of network connectivity).

Network 458 may be implemented as, for example, the internet, a local area network, a virtual network, a wireless network, a cellular network, a local bus, a point-to-point link (e.g., wired), and/or combinations of the aforementioned.

It is noted that data interface(s) 420, data interface(s) 422, and network interface 462 may be implemented as different individual interfaces, and/or one or more combined interfaces.

Computing device 450 may communicate with one or more server(s) 464 over network 458, for example, to obtain other images from other imager(s) via another server, to obtain updated versions of code, and the like.

Computing device 450 may include and/or be in communication with one or more physical user interfaces 404 that include provide a mechanism to enter data (e.g., annotation of training dataset) and/or view data (e.g., enhanced image, indication of state of marker) for example, one or more of, a touchscreen, a display, gesture activation devices, a keyboard, a mouse, and voice activated software using speakers and microphone. Display 406 may be integrated with user interface 404, or be a separate device.

Referring now back to FIG. 5, a computer-implemented method of training a ML model for determining an orientation of an aortic valve prosthesis for trans-catheter deployment depicted in a medical image, and/or for generating an enhanced image, is described. It is noted that the aortic valve prosthesis is a not necessarily limiting example, as other medical devices of other trans-catheter procedures may be depicted. It is noted that other features related to training of the ML model are described herein.

At 502, a sample original medical image depicting the aortic valve prosthesis with a marker, within the body of a subject, is obtained. For example, a 2D fluoroscopic image depicting the valve in the aorta during an aortic valve replacement trans-catheter procedure.

At 504, a pose of the imager that captured the sample medical image may be is obtained. The pose may be obtained by applying an optical character recognition process to the sample medical image, and extracting the automatically recognized characters. Examples of poses include LAO and RAO.

At 506, a region of interest (ROI) of the sample original medical image that depicts the marker is defined. The ROI may be, for example, a frame having dimensions smaller than the sample medical image. The ROI may be sized for depicting the marker, at least a portion of the aortic valve prosthesis, and tissues in proximity to the aortic valve prosthesis, for example, the blood vessel within which the valve is located, and/or the aortic annulus, and the like. Other details of the ROI are described herein.

At 508, an enhanced medical image is created from the ROI, for example, by applying image processing and/or machine learning, for example, as described herein. The enhanced medical image may exclude the portion of the original image external to the ROI. The enhanced medical image may be of a higher quality than the ROI of the sample medical image. The enhanced medical image may be an enlargement of the ROI of the sample medical image. Additional exemplary details of computing the enhanced medical image and/or of the enhanced medical image are described herein.

At 510, an orientation of the aortic valve prosthesis depicted in the enhanced medical image is obtained, for example, manually by a user and/or by a process that analyzes the marker (e.g., pattern of markers) depicted in the enhanced medical image, for example, described herein. It is noted that training the ML model may improve upon using the process that analyzes the marker, for example, the ML model may be of higher accuracy than the process based on learning from many images, and/or the ML model may be able to determine the orientation in view of noise and/or poor image quality where the process that analyzes the marker may fail or be inaccurate.

The orientation of the aortic valve prosthesis may be one or more of the following: whether the orientation of the medical device is proper or not, whether the marker is aligned with the native commissure of the aortic annulus where the aortic valve prosthesis is to be deployed, an angle, and a classification category. Exemplary classification categories include one of four states: outer curve, inner curve, middle front, or middle back. In another example, classification categories include inner curve state, outer curve state, and middle state. The number of the available (distinguishable) states used for classification categories may depend on the kind of prosthetic device used, and it is usually between two and four. Other classification categories are as described herein, for example, for three markers located in the commissures of the prosthetic heart valve, for a single main marker, indicating whether the orientation indicates that the commissures of the prosthetic heart valve are non-aligned with the coronary ostia, and the like.

At 512, a record may be created. The record includes the sample original medical image, and a ground truth. The ground truth may be an indication of the orientation and/or the enhanced medical image. Examples of classification categories of the orientation are described herein, for example, a binary classification indicating correct alignment (e.g., the commissures of the prosthetic valve will not aligned with the coronary ostia and are not predicted to block blood flow into the coronary arteries) or incorrect alignment (e.g., the commissures of the prosthetic valve will aligned with the coronary ostia) and may block blood flow into the coronary arteries. The ground truth may be selected according to the desired outcome of the ML model. The record may further include the pose. The ML model may generate the outcome in response to a further input of the pose. The pose may increase accuracy of the ML model's determination of the orientation, since the same marker on the valve appears differently in the different poses of the imager (e.g., LAO, RAO of the fluoroscopy machine).

At 514, one or more features described with reference to 502-512 may be iterated for each sample medical image of multiple sample original medical images of multiple subjects. Alternatively or additionally, the iterations are for multiple sample original medical images of the same subject, at different times during the procedure, such as when the valve is located at different regions along the aorta. Alternatively or additionally, the iterations are for different images which are created from a certain original medical image, for example, by translation and/or rotation of the medical image, as described herein.

Optionally, individual frames captured during a transcatheter delivery of a prosthetic aortic valve are used to generate a training record, for example, about 300 frames per movie.

Inventors found that images from at least about 30-80 aortic valve replacement procedures may be required to sufficiently train the ML model(s). About 300 suitable images may be obtained from each procedure.

Since movies of training procedures may be limited in supply, additional augmented images may be obtained, for example, by rotation and/or translation and/or zoom and/or other data augmentation approaches, for generating additional training records.

It may be difficult to obtain images in which the markers are visible clearly enough to accurately determine orientation of the valve, for example, weak signal on significant noise and/or variability while approaching and engaging the aortic arch. As such, synthetic images reproducing a realistic pattern similar to best practices examples may be generated and used in training records. A large number of synthetic images may be generated, for example, thousands, covering the different variations and/or noise, which may be used to train a robust ML model.

At 516, a training dataset that includes the multiple records may be created.

At 518, the ML model is trained on the training dataset. The trained ML model generates an outcome of the orientation and/or the enhanced image in response to an input of a target original image depicting a target aortic valve prosthesis for trans-catheter deployment, and optionally the fluoroscopic view of the target original image.

In an alternative architecture, the ML model may include a first ML model component and a second ML model component. Training datasets for such architecture may include creating a first training dataset and a second training dataset. The first dataset includes first records. Each first record includes the sample original medical image and a ground truth of the enhanced medical image. The second training dataset includes multiple second records. Each second record includes the enhanced medical image and a ground truth of the orientation. The first ML model component is trained on the first training dataset, and the second ML model component is trained on the second training dataset. The trained first ML model generates an outcome of the enhanced medical image in response to the input of the target original image;. The trained second ML model component generates an outcome of the orientation in response to an input of the enhanced medical image generated by the first ML model.

In an alternative implementation, the ROI of the sample original image (e.g., as in 506) includes a first boundary (e.g., box) encompassing an entirety of the aortic valve prosthesis. The enhanced medical image (e.g., as in 508) includes a portion of the sample original image within a second boundary located within the first boundary, for example, a smaller box within a larger box. The second boundary encompasses the marker and a portion of the aortic valve prosthesis in proximity to the marker and excluding a remainder of the aortic valve prosthesis. For example, the second boundary box encloses about a third of the valve with the marker being approximately centered in the second boundary box.

In yet another alternative architecture, the ML model may include a first ML model, component, a second ML model component, and a third ML model component. Training datasets for such architecture may include creating a first training dataset, a second training dataset, and a third training dataset. The first training dataset may include first records. Each first record includes the sample original medical image and a ground truth of the first boundary encompassing an entirety of the aortic valve prosthesis. The second training dataset includes second records. Each second record includes the portion of the sample original image within the first boundary and a ground truth of the second boundary. The third training dataset includes third records. Each third record includes a portion of the sample original image within the second boundary and a ground truth of the orientation. Examples of classification categories of the orientation are described herein. The first ML model component is trained on the first training dataset for generating an outcome of the first boundary in response to the input of the target original image. The second ML model component is trained on the second training dataset for generating an outcome of the second boundary in response to the input of the first boundary generated by the first ML. The third ML model component is trained on the third training dataset for generating an outcome of the orientation in response to an input of the portion of the target original medical image within the second boundary generated by the second ML model.*

The ML model(s) may be validated on new, labeled images, and performance of the ML model(s) may be measured. When the results of the validation are satisfactory, the ML model(s) may be used for inference of new images.

Referring now back to FIG. 9, the schematic depicts sample original image 902, first boundary box 904, second boundary box 906, and enhanced image 908 which is labelled with the ground truth of orientation of the valve prosthesis according to the depicted marker, as described herein.

Reference is now made to FIG. 10, which is a schematic 1002 of an exemplary neural network architecture of ML model(s), in accordance with some embodiments of the present invention. The schematic represents a convolutional neural network (CNN) which may be used for one or more of the ML models described herein and/or for components of the ML models described herein (e.g., first, second, third of different embodiments). The details of the architecture may be fine-tuned to obtain improved performance, for example, by trial and error. Each hidden layer may include two distinct stages: the first stage kernel has trainable weights and gets the result of a local convolution of the previous layer. The second stage may be a max-pooling, where the number of parameters is significantly reduced by keeping only the maximum response of several units of the first stage. After several hidden layers, the final layer may be a fully connected layer. It may have a unit for each class that the network predicts, and each of those units receives input from all units of the previous layer. Each CNN may have 4 output units.

The CNN may include one input channel to Conv2D (for fluoroscopic images, which include black and white pixels, i.e., no color). The last layer may be fully connected. The output features may be reduced to 4 to correspond to the four coordinates to predict the bounding box (e.g., x1,y2 represent the upper left corner and x2,y2 represent the lower right corner). An Adam optimizer may be used. The loss function used may be Mean Square Error, to predict distances. A robust framework such as PyTorch may be used to enable focusing on implementing the specific details while the library handles the mechanics of the neural network training in an optimized manner.

At 520, the trained ML model(s) is provided for inference, for example, for generating a presentation for guiding a medical procedure. An exemplary inference process is now described. A processor feeds an original medical image depicting at least a portion of an aortic valve prosthesis with marker for trans-catheter deployment into the trained ML model(s). The processor may further obtain a pose of the original image and feed the pose into the trained ML model(s) in combination with the original medical image. The processor obtains an orientation of the aortic valve prosthesis as an outcome of the ML model(s). Alternatively or additionally, an enhanced image comprising a ROI of the original medical image that depicts the marker and the at least the portion of the aortic valve prosthesis, may be obtained as the outcome of the ML model(s). The processor may generate instructions for presenting the enhanced image as an inset of the target original medical image presented on display, and for presenting the orientation of the aortic valve prosthesis.

Referring now back to FIG. 6, a schematic depicting exemplary orientations of aortic valve prosthesis 602, is depicted. Aortic valve prosthesis 602 includes three markers (604, 606, 608), which may be placed at the commissures of the aortic valve prosthesis. The three markers may appear in different orientations in 2D images (e.g., fluoroscopic images), which may be detected automatically as described herein. For example, the different orientations may be classification categories outputted by ML model(s) in response to an input of a 2D fluoroscopic image. Schematic 610 depicts markers 604 and 606 overlapping on the left side, where marker 608 is non-overlapping; which may represent a correct orientation where the commissures of the valve are non-aligned with the coronary ostia. Schematic 612 depicts non-overlap between any of markers 604, 606 and 608. Schematic 614 depicts markers 606 and 6068 overlapping on the right side, where marker 604 is non-overlapping. The state depicted by 610 is sometimes referred to herein as inner-overlap. The state depicted by 612 is sometimes referred to herein as separate. The state depicted by 614 is sometimes referred to herein as outer-overlap. Schematics 612 and 614 may represent an incorrect orientation where the commissures of the valve are aligned with the coronary ostia (which may block blood flow to the coronary arteries).

Referring now back to FIG. 7, schematics of different orientations of an aortic valve prosthesis that includes three markers located at the commissures of the aortic valve prosthesis, are depicted. Schematics 702, 704, 714 and 718 depict the outer-overlap orientation. Schematics 706, 712, depict the inner-overlap orientation. Schematics 708, 710, 716 depict the separate orientation. Schematics 702-716 may be used, for example, as training images for training ML models as described herein, and/or may be examples of images which are analyzed to determine the orientation, as described herein. Schematics 750 and 752 depicts an arrangement of the commissures of the valve for clarity, which correspond to a certain orientation detected based on an analysis of the markers, as described herein. Schematic 750 depicts the outer-overlap orientation, which may be the correct orientation for deployment. Schematic 752 depicts the inner-overlap orientation, which may be the incorrect orientation for deployment.

Referring now back to FIG. 8, schematics of different orientations of an aortic valve prosthesis (Porticco™ by Abbott) that includes a single main marker 802, shaped approximately as an L, where the long line of the L is approximately horizontal (i.e., “--\hat{ }”), are depicted. The single main marker is shown for clarity in schematics 850 and 852. Marker 850 indicates the outer orientation, which may be the correct orientation for deployment. Marker 852 indicates the inner orientation, which may be the incorrect orientation for deployment. Schematics 804, 808, 810, 812, 816, and 820 represent the outer orientation of the prosthetic heart valve detected according to the appearance of main marker 802, for different poses of main marker 802, for example, different amounts of rotation. Schematics 806 and 818 represent the inner orientation of the prosthetic heart valve detected according to the appearance of main marker 802, for different poses of main marker 802, for example, different amounts of rotations. It is noted that in the inner orientations, main marker 802 may appear as a mirror image of the depiction of main marker 802 in images depicting the outer orientation. Schematics 814 represent the central orientation of the prosthetic heart valve detected according to the appearance of main marker 802.

It is expected that during the life of a patent maturing from this application many relevant implantable and/or endolumenally operated medical devices will be developed; the scope of the term implantable and/or endolumenally operated medical devices is intended to include all such new technologies a priori.

As used herein with reference to quantity or value, the term “about” means “within ±10% of”. The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

In addition, any priority document(s) of this application is/are hereby incorporated herein by reference in its/their entirety.

1. A computer implemented method for generating a presentation of at least one image for assisting an operator in bringing a medical implement having a marker to a target location within a body of a patient, the method comprising:
receiving a first image of the medical implement in the body of the patient, the implement being on its way to the target location;
processing the first image to obtain an enhanced image showing at least said marker; and
providing the first image and the enhanced image for display, wherein the enhanced image is provided for display as an inset on a display of the first image.

2. A computer implemented method for generating a presentation of at least one image for assisting an operator in bringing a medical implement having a marker to a target location within a body of a patient, the method comprising:
receiving a first image of the medical implement in the body of the patient, the implement being on its way to the target location;
processing the first image to obtain an enhanced image of a region of interest showing at least said marker; and
providing the enhanced image for display,
wherein the processing comprises inputting the first image to a machine-learning model trained to identify the region of interest encompassing at least said marker.

3. The computer implemented method of claim 1, wherein said processing comprises
identifying a portion of the first image as a region of interest comprising the marker;
cropping the identified portion from the first image; and
enhancing the cropped portion of the first image.

4. The computer implemented method of claim 1, wherein the enhanced image shows a portion of the medical implement enlarged in comparison to its size in the first image.

5. The computer implemented method of claim 1, further comprising
estimating a roll angle of the medical implement based on appearance of the marker in the first or enhanced image, and
providing for display an indication to the estimated roll angle.

6. The computer implemented method of claim 5, wherein the marker is shaped to display on the first image a portion of its length depending on the roll angle, and the processing comprises estimating the roll angle based on the length of the marker shown in the first or enhanced image.

7. The computer implemented method of claim 5, wherein estimating the roll angle comprises identifying spatial relationships between marking elements of the marker shown in the first or enhanced image.

8. The computer implemented method of claim 3, wherein the processing comprises inputting the first image to a machine-learning model trained to identify the region of interest encompassing at least said marker.

9. The computer implemented method of claim 5, wherein the processing comprises inputting the first image to a machine-learning model trained to estimate the roll angle.

10. The computer implemented method of claim 1, repeated at least 10 times per second, to provide for display of a cine of enhanced images.

11. The computer implemented method of claim 1, wherein the first image is a fluoroscopic image.

12. A system for generating a presentation of at least one image for assisting an operator in bringing a medical implement having a marker to a target location within a body of a patient, the system comprising:
a memory, storing instructions;
a processor, configured to execute the instructions,
wherein executing the instructions cause the processor to
receive a first image of the medical implement;
process the first image to obtain an enhanced image showing at least said marker; and
provide the first image and the enhanced image for display, wherein the enhanced image is provided for display as an inset on a display of the first image.

13. A system for generating a presentation of at least one image for assisting an operator in bringing a medical implement having a marker to a target location within a body of a patient, the system comprising:
a memory, storing instructions; and
a processor, configured to execute the instructions,
wherein executing the instructions cause the processor to:
receive a first image of the medical implement in the body of the patient, the implement being on its way to the target location;
apply to the first image a machine-learning model trained to identify a region of interest encompassing at least said marker;
process the first image to obtain an enhanced image of the region of interest showing at least said marker; and
provide the enhanced image for display to the operator.

14. The system of claim 12, wherein the instructions cause the processor to
identify a portion of the first image as a region of interest comprising the marker;
crop the identified portion from the first image; and
enhance at least the cropped portion of the first image.

15. The system of claim 12, wherein the enhanced image shows a portion of the medical implement enlarged in comparison to a size of said portion of the medical implement in the first image.

16. The system of claim 12, wherein the instructions further cause the processor to
estimate a roll angle of the medical implement based on the appearance of the mark in the first or enhanced image, and
provide for display an indication to the estimated roll angle.

17. The system of claim 16, wherein the instructions cause the processor to estimate the roll angle based on the length of the marker shown in the first or enhanced image.

18. The system of claim 16, wherein the instructions cause the processor to estimate the roll angle by identifying spatial relationships between marking elements of the marker shown in the first or enhanced image.

19. The system of claim 14, wherein the instructions cause the processor to apply, to the first image or to the enhanced image, a machine-learning model trained to identify the region of interest encompassing at least said marker.

20. The system of claim 16, the instructions cause the processor to apply, to the first image or to the enhanced image, a machine-learning model trained to estimate the roll angle.

21. The system of claim 14, wherein the instructions cause the processor to repeat the receiving, processing, and providing for display at least 10 times per second, to provide for display of a cine of enhanced images.

22. The system of claim 12, wherein the first image is a fluoroscopic image.

23. (canceled)

24. The system of claim 12, wherein the instructions cause the processor to provide the enhanced image for display to an output connected to a display of the imaging device.

25-91. (canceled)
